(19) United States
(12) Patent Application Publication
(10) Pub. No.: US 2025/0241961 A1
(43) Pub. Date: Jul. 31, 2025

(54) STEM CELL IMMUNOTHERAPY FOR COLITIS

(71) Applicant: SPHERE THERAPEUTICS, INC., Hollywood, FL (US)

(72) Inventors: Thomas ICHIM, San Diego, CA (US); Michael GURMAN, Hollywood, FL (US); James D Veltmeyer, La Jolla, CA (US); Roman A Ramos, San Diego, CA (US)

(21) Appl. No.: 19/037,726

(22) Filed: Jan. 27, 2025

Related U.S. Application Data
(60) Provisional application No. 63/624,976, filed on Jan. 25, 2024.

(51) Int. Cl. A61K 35/28; A61P 1/00; C12N 5/0775

(52) U.S. Cl. CPC A61K 35/28; A61P 1/00; C12N 5/0665; C12N 2501/24

(57) ABSTRACT

Disclosed are compositions of matter, therapeutic interventions, and combination treatments for preventing and/or reducing colitis. In one embodiment, administration of specific mesenchymal stem cell subsets into a patient suffering from colitis, wherein said mesenchymal stem cells possess enhanced ability to stimulate T regulatory cells. In one embodiment the patient is administered a composition capable of altering the colonic microenvironment prior to administration of stem cells. In other embodiments microbiome associated metabolites are administered prior to, concurrent with, or subsequent to stem cell administration. In some embodiments treatment of colitis is accomplished by stimulation of immune modulation together with mesenchymal stem cell administration.

### CROSS-REFERENCE TO RELATED APPLICATIONS

This application claims the benefit of U.S. Provisional Application No. 63/624,976 filed on Jan. 25, 2024, which is incorporated by reference in its entirety.

### BACKGROUND

The present disclosure related to means of treating autoimmunity and ulcerative colitis through modulation of the immune system.

When the immune system of an organism produces a response against its own healthy cells, it may lead to an autoimmune disease. Ulcerative colitis is an autoimmune disease that causes inflammation in the large intestine and/or rectum. Treatment options may include stem cells that are able to modulate the immune system.

### SUMMARY

In aspects, the disclosure relates to methods of preventing and/or treating colitis. In some embodiments, the method includes administering a population of mesenchymal stem cells possessing enhanced ability to induce generation of T regulatory cells as compared to conventional mesenchymal stem cells. In some embodiments, the method includes administering separately or together a cell-free means or composition possessing therapeutic potential. In some embodiments, the colitis is mediated by an autoimmune response in patient suffering from said colitis. In some embodiments, the autoimmune response is associated with activation of Th17 cells. In some embodiments, the Th17 cells are associated with reduced generation of T regulatory cells. In some embodiments, the Th17 cells are associated with reduced generation of T regulatory 10 (Tr10) cells. In some embodiments, the T regulatory cells express FoxP3. In some embodiments, the T regulatory cells express Fas ligand. In some embodiments, the T regulatory cells express granzyme B. In some embodiments, the T regulatory cells express perforin. In some embodiments, the T regulatory cells express membrane bound TGF-beta. In some embodiments, the T regulatory cells suppress dendritic cell maturation.

In some embodiments, the dendritic cell maturation is associated with upregulation of interleukin-12 production. In some embodiments, the dendritic cell maturation is associated with upregulation of interleukin-1 beta production. In some embodiments, the dendritic cell maturation is associated with upregulation of interleukin-2 production. In some embodiments, the dendritic cell maturation is associated with upregulation of interleukin-6 production. In some embodiments, the dendritic cell maturation is associated with upregulation of TNF-alpha production. In some embodiments, the dendritic cell maturation is associated with upregulation of lymphotoxin production. In some embodiments, the dendritic cell maturation is associated with upregulation of TRAIL production. In some embodiments, the dendritic cell maturation is associated with upregulation of TWEAK production. In some embodiments, the dendritic cell maturation is associated with down regulation of interleukin-10 production. In some embodiments, the dendritic cell maturation is associated with down regulation of interleukin-12 p40 homodimer production. In some embodiments, the dendritic cell maturation is associated with down regulation of soluble HLA-G production. In some embodiments, the dendritic cell maturation is associated with down regulation of soluble TNF-alpha receptor p55 production. In some embodiments, the dendritic cell maturation is associated with down regulation of soluble TNF-alpha receptor p75 production. In some embodiments, the dendritic cell maturation is associated with down regulation of soluble TNF-alpha receptor p55: p75 heterodimer production. In some embodiments, the dendritic cell maturation is associated with upregulation of CD5 expression. In some embodiments, the dendritic cell maturation is associated with upregulation of CD40 expression. In some embodiments, the dendritic cell maturation is associated with upregulation of CD80 expression. In some embodiments, the dendritic cell maturation is associated with upregulation of CD86 expression. In some embodiments, the dendritic cell maturation is associated with upregulation of HLA II expression.

In some embodiments, the mesenchymal stem cells possessing enhanced ability to induce generation of T regulatory cells as compared to conventional mesenchymal stem cells are autologous to the patient being treated. In some embodiments, the mesenchymal stem cells possessing enhanced ability to induce generation of T regulatory cells as compared to conventional mesenchymal stem cells are allogeneic to the patient being treated. In some embodiments, the mesenchymal stem cells possessing enhanced ability to induce generation of T regulatory cells as compared to conventional mesenchymal stem cells are xenogeneic to the patient being treated. In some embodiments, the mesenchymal stem cells possessing enhanced ability to induce generation of T regulatory cells as compared to conventional mesenchymal stem cells are derived from perinatal tissue. In some embodiments, the perinatal tissue derived mesenchymal stem cells are obtained from Wharton's Jelly. In some embodiments, the perinatal tissue derived mesenchymal stem cells are obtained from subepithelial cord tissue. In some embodiments, the Wharton's Jelly mesenchymal stem cell is plastic adherent. In some embodiments, the Wharton's Jelly mesenchymal stem cell is collagen II adherent. In some embodiments, the Wharton's Jelly mesenchymal stem cell is fibronectin adherent. In some embodiments, the Wharton's Jelly mesenchymal stem cell is adherent to hyaluronic acid. In some embodiments, the Wharton's Jelly mesenchymal stem cell is aggrecan adherent. In some embodiments, the Wharton's Jelly mesenchymal stem cell is selected for enhanced expression of CD56 as compared to other mesenchymal stem cells from Wharton's Jelly. In some embodiments, the cells with enhanced expression of CD56 are selected after culture of Wharton's Jelly derived mesenchymal stem cells for at least 24 hours with interferon gamma. In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, the cells with enhanced expression of CD56 are selected after culture of Wharton's Jelly derived mesenchymal stem cells for at least 48 hours with interferon gamma. In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, the cells with enhanced expression of CD56 are selected after culture of Wharton's Jelly derived mesenchymal stem cells for at least 72 hours with interferon gamma. In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, cells with enhanced expression of CD73 are selected after culture of Wharton's Jelly derived mesenchymal stem cells for at least 24 hours with interferon gamma. In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, cells with enhanced expression of CD73 are selected after culture of Wharton's Jelly derived mesenchymal stem cells for at least 48 hours with interferon gamma. In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, cells with enhanced expression of CD73 are selected after culture of Wharton's Jelly derived mesenchymal stem cells for at least 72 hours with interferon gamma.

In some embodiments, the concentration of interferon gamma is sufficient to enhance expression of HLA II at least 50% as compared to baseline. In some embodiments, the concentration of interferon gamma is sufficient to enhance expression of HLA II at least 100% as compared to baseline. In some embodiments, the concentration of interferon gamma is sufficient to enhance expression of HLA II at least 200% as compared to baseline. In some embodiments, Wharton's Jelly derived mesenchymal stem cell is cultured under conditions to enhance expression of HLA-G. In some embodiments, culture conditions to increase expression of HLA-G are exposure to interleukin-10 at a concentration and duration to suppress interferon induced expression of HLA II by over 25% as compared to baseline expression of HLA II induced by interferon gamma. In some embodiments, the interferon gamma is administered at a concentration of 1-10,000 IU/million mesenchymal stem cells. In some embodiments, the interferon gamma is administered at a concentration of 1-1,000 IU/million mesenchymal stem cells. In some embodiments, the interferon gamma is administered at a concentration of 10-100 IU/million mesenchymal stem cells. In some embodiments, culture conditions to increase expression of HLA-G are exposure to interleukin-10 at a concentration and duration to suppress interferon induced expression of HLA II by over 50% as compared to baseline expression of HLA II induced by interferon gamma. In some embodiments, culture conditions to increase expression of HLA-G are exposure to interleukin-10 at a concentration and duration to suppress interferon induced expression of HLA II by over 75% as compared to baseline expression of HLA II induced by interferon gamma. In some embodiments, culture conditions to increase expression of HLA-G are exposure to interleukin-4 at a concentration and duration to suppress interferon induced expression of HLA II by over 25% as compared to baseline expression of HLA II induced by interferon gamma. In some embodiments, culture conditions to increase expression of HLA-G are exposure to interleukin-4 at a concentration and duration to suppress interferon induced expression of HLA II by over 50% as compared to baseline expression of HLA II induced by interferon gamma. In some embodiments, culture conditions to increase expression of HLA-G are exposure to interleukin-4 at a concentration and duration to suppress interferon induced expression of HLA II by over 75% as compared to baseline expression of HLA II induced by interferon gamma. In some embodiments, culture conditions to increase expression of HLA-G are exposure to interleukin-13 at a concentration and duration to suppress interferon induced expression of HLA II by over 25% as compared to baseline expression of HLA II induced by interferon gamma. In some embodiments, culture conditions to increase expression of HLA-G are exposure to interleukin-13 at a concentration and duration to suppress interferon induced expression of HLA II by over 50% as compared to baseline expression of HLA II induced by interferon gamma. In some embodiments, culture conditions to increase expression of HLA-G are exposure to interleukin-13 at a concentration and duration to suppress interferon induced expression of HLA II by over 75% as compared to baseline expression of HLA II induced by interferon gamma. In some embodiments, culture conditions to increase expression of HLA-G are exposure to interleukin-20 at a concentration and duration to suppress interferon induced expression of HLA II by over 25% as compared to baseline expression of HLA II induced by interferon gamma. In some embodiments, culture conditions to increase expression of HLA-G are exposure to interleukin-20 at a concentration and duration to suppress interferon induced expression of HLA II by over 50% as compared to baseline expression of HLA II induced by interferon gamma. In some embodiments, culture conditions to increase expression of HLA-G are exposure to interleukin-20 at a concentration and duration to suppress interferon induced expression of HLA II by over 75% as compared to baseline expression of HLA II induced by interferon gamma. In some embodiments, culture conditions to increase expression of HLA-G are exposure to interleukin-22 at a concentration and duration to suppress interferon induced expression of HLA II by over 25% as compared to baseline expression of HLA II induced by interferon gamma. In some embodiments, culture conditions to increase expression of HLA-G are exposure to interleukin-22 at a concentration and duration to suppress interferon induced expression of HLA II by over 50% as compared to baseline expression of HLA II induced by interferon gamma. In some embodiments, culture conditions to increase expression of HLA-G are exposure to interleukin-22 at a concentration and duration suppress interferon induced expression of HLA II by over 75% as compared to baseline expression of HLA II induced by interferon gamma. In some embodiments, culture conditions to increase expression of HLA-G are exposure to interleukin-35 at a concentration and duration to suppress interferon induced expression of HLA II by over 25% as compared to baseline expression of HLA II induced by interferon gamma. In some embodiments, culture conditions to increase expression of HLA-G are exposure to interleukin-35 at a concentration and duration to suppress interferon induced expression of HLA II by over 50% as compared to baseline expression of HLA II induced by interferon gamma. In some embodiments, culture conditions to increase expression of HLA-G are exposure to interleukin-35 at a concentration and duration to suppress interferon induced expression of HLA II by over 75% as compared to baseline expression of HLA II induced by interferon gamma. In some embodiments, culture conditions to increase expression of HLA-G are exposure to interleukin-37 at a concentration and duration to suppress interferon induced expression of HLA II by over 25% as compared to baseline expression of HLA II induced by interferon gamma. In some embodiments, culture conditions to increase expression of HLA-G are exposure to interleukin-37 at a concentration and duration to suppress interferon induced expression of HLA II by over 50% as compared to baseline expression of HLA II induced by interferon gamma. In some embodiments, culture conditions to increase expression of HLA-G are exposure to interleukin-37 at a concentration and duration to suppress interferon induced expression of HLA II by over 75% as compared to baseline expression of HLA II induced by interferon gamma. In some embodiments, culture conditions to increase expression of HLA-G are exposure to interleukin-38 at a concentration and duration to suppress interferon induced expression of HLA II by over 25% as compared to baseline expression of HLA II induced by interferon gamma. In some embodiments, culture conditions to increase expression of HLA-G are exposure to interleukin-38 at a concentration and duration to suppress interferon induced expression of HLA II by over 50% as compared to baseline expression of HLA II induced by interferon gamma.

In some embodiments, culture conditions to increase expression of HLA-G are exposure to interleukin-38 at a concentration and duration to suppress interferon induced expression of HLA II by over 75% as compared to baseline expression of HLA II induced by interferon gamma.

In some embodiments, culture conditions to increase expression of HLA-G are exposure to VEGF at a concentration and duration to suppress interferon induced expression of HLA II by over 25% as compared to baseline expression of HLA II induced by interferon gamma. In some embodiments, culture conditions to increase expression of HLA-G are exposure to VEGF at a concentration and duration to suppress interferon induced expression of HLA II by over 50% as compared to baseline expression of HLA II induced by interferon gamma. In some embodiments, culture conditions to increase expression of HLA-G are exposure to VEGF at a concentration and duration to suppress interferon induced expression of HLA II by over 75% as compared to baseline expression of HLA II induced by interferon gamma.

In some embodiments, culture conditions to increase expression of HLA-G are exposure to EGF at a concentration and duration to suppress interferon induced expression of HLA II by over 25% as compared to baseline expression of HLA II induced by interferon gamma. In some embodiments, culture conditions to increase expression of HLA-G are exposure to EGF at a concentration and duration to suppress interferon induced expression of HLA II by over 50% as compared to baseline expression of HLA II induced by interferon gamma. In some embodiments, culture conditions to increase expression of HLA-G are exposure to EGF at a concentration and duration to suppress interferon induced expression of HLA II by over 75% as compared to baseline expression of HLA II induced by interferon gamma.

In some embodiments, culture conditions to increase expression of HLA-G are exposure to FGF-1 at a concentration and duration suppress interferon induced expression of HLA II by over 25% as compared to baseline expression of HLA II induced by interferon gamma. In some embodiments, culture conditions to increase expression of HLA-G are exposure to FGF-1 at a concentration and duration to suppress interferon induced expression of HLA II by over 50% as compared to baseline expression of HLA II induced by interferon gamma. In some embodiments, culture conditions to increase expression of HLA-G are exposure to FGF-1 at a concentration and duration to suppress interferon induced expression of HLA II by over 75% as compared to baseline expression of HLA II induced by interferon gamma. In some embodiments, culture conditions to increase expression of HLA-G are exposure to FGF-2 at a concentration and duration to suppress interferon induced expression of HLA II by over 25% as compared to baseline expression of HLA II induced by interferon gamma. In some embodiments, culture conditions to increase expression of HLA-G are exposure to FGF-2 at a concentration and duration to suppress interferon induced expression of HLA II by over 50% as compared to baseline expression of HLA II induced by interferon gamma. In some embodiments, culture conditions to increase expression of HLA-G are exposure to FGF-2 at a concentration and duration to suppress interferon induced expression of HLA II by over 75% as compared to baseline expression of HLA II induced by interferon gamma. In some embodiments, culture conditions to increase expression of HLA-G are exposure to FGF-5 at a concentration and duration to suppress interferon induced expression of HLA II by over 25% as compared to baseline expression of HLA II induced by interferon gamma. In some embodiments, culture conditions to increase expression of HLA-G are exposure to FGF-5 at a concentration and duration to suppress interferon induced expression of HLA II by over 50% as compared to baseline expression of HLA II induced by interferon gamma. In some embodiments, culture conditions to increase expression of HLA-G are exposure to FGF-5 at a concentration and duration to suppress interferon induced expression of HLA II by over 75% as compared to baseline expression of HLA II induced by interferon gamma.

In some embodiments, culture conditions to increase expression of HLA-G are exposure to HGF at a concentration and duration to suppress interferon induced expression of HLA II by over 25% as compared to baseline expression of HLA II induced by interferon gamma. In some embodiments, culture conditions to increase expression of HLA-G are exposure to HGF at a concentration and duration to suppress interferon induced expression of HLA II by over 50% as compared to baseline expression of HLA II induced by interferon gamma. In some embodiments, culture conditions to increase expression of HLA-G are exposure to HGF at a concentration and duration to suppress interferon induced expression of HLA II by over 75% as compared to baseline expression of HLA II induced by interferon gamma. In some embodiments, culture conditions to increase expression of HLA-G are exposure to interferon beta at a concentration and duration to suppress interferon induced expression of HLA II by over 25% as compared to baseline expression of HLA II induced by interferon gamma. In some embodiments, culture conditions to increase expression of HLA-G are exposure to interferon beta at a concentration and duration to suppress interferon induced expression of HLA II by over 50% as compared to baseline expression of HLA II induced by interferon gamma. In some embodiments, culture conditions to increase expression of HLA-G are exposure to interferon beta at a concentration and duration to suppress interferon induced expression of HLA II by over 75% as compared to baseline expression of HLA II induced by interferon gamma. In some embodiments, culture conditions to increase expression of HLA-G are exposure to angiopoietin at a concentration and duration to suppress interferon induced expression of HLA II by over 25% as compared to baseline expression of HLA II induced by interferon gamma. In some embodiments, culture conditions to increase expression of HLA-G are exposure to angiopoietin at a concentration and duration to suppress interferon induced expression of HLA II by over 50% as compared to baseline expression of HLA II induced by interferon gamma. In some embodiments, culture conditions to increase expression of HLA-G are exposure to angiopoietin at a concentration and duration to suppress interferon induced expression of HLA II by over 75% as compared to baseline expression of HLA II induced by interferon gamma. In some embodiments, culture conditions to increase expression of HLA-G are exposure to placental growth factor at a concentration and duration to suppress interferon induced expression of HLA II by over 25% as compared to baseline expression of HLA II induced by interferon gamma. In some embodiments, culture conditions to increase expression of HLA-G are exposure to placental growth factor at a concentration and duration to suppress interferon induced expression of HLA II by over 50% as compared to baseline expression of HLA II induced by interferon gamma. In some embodiments, culture conditions to increase expression of HLA-G are exposure to placental growth factor at a concentration and duration to suppress interferon induced expression of HLA II by over 75% as compared to baseline expression of HLA II induced by interferon gamma. In some embodiments, culture conditions to increase expression of HLA-G are exposure to progesterone induced blocking factor at a concentration and duration to suppress interferon induced expression of HLA II by over 25% as compared to baseline expression of HLA II induced by interferon gamma. In some embodiments, culture conditions to increase expression of HLA-G are exposure to progesterone induced blocking factor at a concentration and duration to suppress interferon induced expression of HLA II by over 50% as compared to baseline expression of HLA II induced by interferon gamma. In some embodiments, culture conditions to increase expression of HLA-G are exposure to progesterone induced blocking factor at a concentration and duration to suppress interferon induced expression of HLA II by over 75% as compared to baseline expression of HLA II induced by interferon gamma. In some embodiments, culture conditions to increase expression of HLA-G are exposure to human chorionic gonadotrophin at a concentration and duration to suppress interferon induced expression of HLA II by over 25% as compared to baseline expression of HLA II induced by interferon gamma. In some embodiments, culture conditions to increase expression of HLA-G are exposure to human chorionic gonadotrophin at a concentration and duration to suppress interferon induced expression of HLA II by over 50% as compared to baseline expression of HLA II induced by interferon gamma. In some embodiments, culture conditions to increase expression of HLA-G are exposure to human chorionic gonadotrophin at a concentration and duration to suppress interferon induced expression of HLA II by over 75% as compared to baseline expression of HLA II induced by interferon gamma. In some embodiments, culture conditions to increase expression of HLA-G are exposure to lithium or a lithium salt at a concentration and duration to suppress interferon induced expression of HLA II by over 25% as compared to baseline expression of HLA II induced by interferon gamma. In some embodiments, culture conditions to increase expression of HLA-G are exposure to lithium or a lithium salt at a concentration and duration to suppress interferon induced expression of HLA II by over 50% as compared to baseline expression of HLA II induced by interferon gamma. In some embodiments, culture conditions to increase expression of HLA-G are exposure to lithium or a lithium salt at a concentration and duration to suppress interferon induced expression of HLA II by over 75% as compared to baseline expression of HLA II induced by interferon gamma.

In some embodiments, the mesenchymal stem cells do not express CD45. In some embodiments, mesenchymal stem cells do not express CD34. In some embodiments, the mesenchymal stem cells do not express HLA-G. In some embodiments, the mesenchymal stem cells are capable of suppressing T cell proliferation. In some embodiments, T cell proliferation is induced by stimulation of said T cells with one or more mitogens. In some embodiments, the mitogen is a lectin. In some embodiments, the mitogen is phytohemagglutinin. In some embodiments, the mitogen is concanavalin A.

In some embodiments, the mitogen is one or more antibodies capable of crosslinking the T cell receptor. In some embodiments, the mitogen is one or more antibodies capable of crosslinking the T cell receptor and one or more costimulatory receptors. In some embodiments, the antibody capable of crosslinking said T cell receptor is an anti-CD3 antibody. In some embodiments, the antibody capable of crosslinking said T cell receptor is an anti-TCR antibody. In some embodiments, the antibody capable of crosslinking said T cell receptor is an anti-CD52 antibody. In some embodiments, the antibody capable of crosslinking a costimulatory molecule is an anti-CD28 antibody. In some embodiments, the antibody capable of crosslinking a costimulatory molecule is an anti-CD40 antibody. In some embodiments, the antibody capable of crosslinking a costimulatory molecule is an anti-CD5 antibody. In some embodiments, the antibody capable of crosslinking a costimulatory molecule is an anti-CD25 antibody. In some embodiments, the antibody capable of crosslinking a costimulatory molecule is an anti-ICAM antibody.

In some embodiments, the cell-free means is a conditioned media. In some embodiments, the cell-free means is a protein concentrate. In some embodiments, the cell-free means is a concentrated nucleic acid composition. In some embodiments, the cell-free means is a concentrated microRNA composition. In some embodiments, the cell-free means is composition of concentrated apoptotic bodies. In some embodiments, the cell-free means is composition of concentrated exosomes. In some embodiments, the cell free means is collected from a pluripotent stem cell. In some embodiments, the pluripotent stem cell is an induced pluripotent stem cell. In some embodiments, the pluripotent stem cell is a dedifferentiated monocyte.

In some embodiments, the monocyte is dedifferentiated by exposure to conditioned media from iPSC. In some embodiments, the conditioned media is generated by culture of iPSC cells in the form of cellular bodies in a liquid media. In some embodiments, the liquid media is Iscove's media. In some embodiments, the liquid media is DMEM media. In some embodiments, the liquid media is OptiMEM media. In some embodiments, the liquid media is EMEM media. In some embodiments, the liquid media is RPMI-1640 media. In some embodiments, the liquid media is AIM-V media.

In some embodiments, the cellular body is disassembled once every 2 days. In some embodiments, the cellular body is disassembled once every 5 days. In some embodiments, the cellular body is disassembled once every 10 days. In some embodiments, the cellular body is composed of iPSC cells together with monocytes. In some embodiments, the monocytes are first treated with a stressor before being admixed with iPSC for generation of conditioned media. In some embodiments, the stressor is hypoxia. In some embodiments, the stressor is hypertonicity. In some embodiments, the stressor is hypotonicity. In some embodiments, the stressor is hyperthermia. In some embodiments, the stressor is serum starvation. In some embodiments, the stressor is mTOR inhibition. In some embodiments, the stressor is AMPK activation. In some embodiments, the stressor is activation of inflammatory pathways.

In some embodiments, the inflammatory pathway is MAP kinase. In some embodiments, the inflammatory pathway is Janus Activated Kinase. In some embodiments, the inflammatory pathway is Signal Transducer and Activator of Transcription-3. In some embodiments, the inflammatory pathway is Signal Transducer and Activator of Transcription-5. In some embodiments, the inflammatory pathway is Signal Transducer and Activator of Transcription-6. In some embodiments, the inflammatory pathway is TLR2. In some embodiments, the TLR2 is activated by peptidoglycan. In some embodiments, the inflammatory pathway is TLR3. In some embodiments, the TLR3 is activated by double stranded RNA. In some embodiments, the TLR3 is activated by Poly IC. In some embodiments, the TLR3 is activated by Poly LC. In some embodiments, the TLR3 is activated by Poly IC: IC. In some embodiments, the TLR3 is activated by inactivated influenza virus.

In some embodiments, the inflammatory pathway is TLR4. In some embodiments, the TLR4 activator are neutrophil extracellular traps. In some embodiments, the TLR4 activator is beta glucan. In some embodiments, the TLR4 activator is HMGB1. In some embodiments, the TLR4 activator is lipopolysaccharide. In some embodiments, the TLR4 activator is yeast cell wall extract. In some embodiments, the TLR4 activator is anti-TLR4 antibody.

In some embodiments, the iPSC cells are engineered to express an inhibitor of TGF-beta. In some embodiments, the inhibitor of TGF-beta is an antibody molecule. In some embodiments, the inhibitor of TGF-beta is a cameloid antibody. In some embodiments, the inhibitor of TGF-beta is a microbody. In some embodiments, the inhibitor of TGF-beta is an aptamer. In some embodiments, the inhibitor of TGF-beta is a molecule capable of inducing the process of RNA interference. In some embodiments, the molecule capable of inducing RNA interference is a short hairpin RNA. In some embodiments, the molecule capable of inducing RNA interference is a short interfering RNA. In some embodiments, the molecule capable of inducing RNA interference is a microRNA. In some embodiments, the molecule capable of inducing RNA interference is a long noncoding RNA.

In some embodiments, the cell-free means is extracted or obtained from a regenerative cell, wherein said regenerative cell has been treated with a “promoting agent”, wherein said promoting agent inhibits cellular differentiation while maintaining pluripotency. In some embodiments, the promoting agent is GDF-11. In some embodiments, the promoting agent is GDF-15. In some embodiments, the promoting agent is amniotic fluid. In some embodiments, the promoting agent is umbilical cord blood plasma. In some embodiments, the promoting agent is BMP2. In some embodiments, the promoting agent suppresses NF-kappa B activation. In some embodiments, the promoting agent increases NRF2 activation. In some embodiments, the promoting agent increases heme-oxygenase-1 activation. In some embodiments, the promoting agent increases bcl-2 activation. In some embodiments, the promoting agent increases bcl-2XL activation. In some embodiments, the promoting agent increases survivin activation. In some embodiments, the promoting agent increases livin activation.

In some embodiments, the promoting agent is an HDAC inhibitor. In some embodiments, the HDAC inhibitor is valproic acid. In some embodiments, the HDAC inhibitor is trichostatin A. In some embodiments, the HDAC inhibitor is sodium phenylbutyrate. In some embodiments, the HDAC inhibitor is butyrate.

In some embodiments, the promoting agent is a GSK-3 inhibitor. In some embodiments, the GSK-3 inhibitor is lithium.

In some embodiments, the promoting agent causes a change in the genotype of the thymic medullary epithelial cell population. In some embodiments, at least one promoting agent comprises an immortalizing oncogene. In some embodiments, at least one promoting agent consists of an immortalizing oncogene. In some embodiments, at least one promoting agent comprises or consists of an immortalizing oncogene. In some embodiments, the promoting agent is PIM1. In some embodiments, the promoting agent is SV40 large T antigen. In some embodiments, the promoting agent is abl1. In some embodiments, the promoting agent is AFF4. In some embodiments, the promoting agent is AKT2. In some embodiments, the promoting agent is AKL. In some embodiments, the promoting agent is AML1. In some embodiments, the promoting agent is MTG8. In some embodiments, the promoting agent is BCL6. In some embodiments, the promoting agent is MCF2. In some embodiments, the promoting agent is DCF3. In some embodiments, the promoting agent is EGFR. In some embodiments, the promoting agent is MLLT11. In some embodiments, the promoting agent is ERBB2. In some embodiments, the promoting agent is ETS1. In some embodiments, the promoting agent is CSFIR. In some embodiments, the promoting agent is FOS. In some embodiments, the promoting agent is FES. In some embodiments, the promoting agent is GNAS. In some embodiments, the promoting agent is HER2. In some embodiments, the promoting agent is FGF3. In some embodiments, the promoting agent is FGF4. In some embodiments, the promoting agent is JUN. In some embodiments, the promoting agent is c-kit. In some embodiments, the promoting agent is K-SAM. In some embodiments, the promoting agent is AKAP13. In some embodiments, the promoting agent is LCK. In some embodiments, the promoting agent is LM01. In some embodiments, the promoting agent is LYL1. In some embodiments, the promoting agent is MAS1. In some embodiments, the promoting agent is MDM2. In some embodiments, the promoting agent is MOS. In some embodiments, the promoting agent is MYH11. In some embodiments, the promoting agent is MYB. In some embodiments, the promoting agent is MYCN. In some embodiments, the promoting agent is PAX5. In some embodiments, the promoting agent is RAF. In some embodiments, the promoting agent is RAS. In some embodiments, the promoting agent is REL. In some embodiments, the said promoting agent is ROS1. In some embodiments, the promoting agent is SKI (PDGF-BB). In some embodiments, the promoting agent is SET. In some embodiments, the promoting agent is SRC. In some embodiments, the promoting agent is TAL1. In some embodiments, the promoting agent is TAN1. In some embodiments, the promoting agent is TIAN. In some embodiments, the promoting agent is TSC2. In some embodiments, the promoting agent is TRK. In some embodiments, the promoting agent can be conditionally inactivated.

### BRIEF DESCRIPTION OF THE DRAWINGS

FIG. 1 shows a non-limiting example of a bar graph of T cell proliferation over time following stimulation with antigen and incubation with Wharton's Jelly mesenchymal stem cells.

FIG. 2 shows a non-limiting example of a bar graph of inflammatory T cell infiltration in mouse colonic tissue (per viewing field) over time.

FIG. 3 shows a non-limiting example of a graph of colitis severity scores.

### DETAILED DESCRIPTION

The disclosure provides means of treating autoimmunity in general and specifically colitis. In some embodiments, the method includes administration of immune modulatory mesenchymal stem cells into a patient suffering from autoimmune processes. In some embodiments specific administration of mesenchymal stem cells is performed into area of diseased colitis inflammation in order to evoke a self-sustaining immunoregulatory process that leads to long term remission and eventual cure of colitis. In some embodiments the disclosure teaches the replication of the processes associated with pregnancy associated remission. In specific embodiments, perinatal tissue is utilized to induce immunological processes associated with pregnancy, specifically the process of fetal escape from immune attack. In one embodiment, the disclosure teaches means of inducing generation of T regulatory cells from perinatal tissue derived exosomes.

The present disclosure is directed to methods for treating autoimmune diseases in general and specifically ulcerative colitis (UC). Certain embodiments concern methods of correcting or ameliorating one or more abnormalities associated with UC in an individual. The individual may be administered perinatal tissue derived exosomes at a concentration and frequency sufficient to correct or ameliorate one or more abnormalities associated with UC. The concentration and frequency may be adjusted based on the response of the individual to the exosomes.

The section headings used herein are for organizational purposes only and are not to be construed as limiting the subject matter described. Features disclosed under one heading (such as a composition) can be used in combination with features disclosed under a different heading (a method of treating). Unless defined otherwise, all technical and scientific terms used herein have the same meaning as is commonly understood by one of ordinary skill in the art. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function of the disclosure, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

As used in the specification and the appended claims, the singular forms “a,” “an” and “the” include plural referents unless the context clearly dictates otherwise. The use of “or” or “and” means “and/or” unless stated otherwise. Furthermore, use of the term “including” as well as other forms, such as “include”, “includes,” and “included,” is not limiting. The mere fact that certain measures are recited in mutually different dependent claims does not indicate that a combination of these measures cannot be used to advantage.

The term “about” or “approximately” means within an acceptable error range for the particular value as determined by one of ordinary skill in the art, which will depend in part on how the value is measured or determined, e.g., the limitations of the measurement system. Where particular values are described in the application and claims, unless otherwise stated, the term “about” means “within an acceptable error range for the particular value.”

In some embodiments, abnormalities associated with UC include an abnormally high level of interleukin (IL)-17 in an individual, an abnormally low level of IL-10 in an individual, and/or an abnormally low level of proliferation of endogenous stem cells in an individual, including endogenous stem cells in the GI tract of the individual. In some embodiments, an abnormally low level of endogenous stem cell proliferation comprises a level of endogenous stem cell proliferation that is 10%, 20%, 30%, 40%, 50%, 60%, 70%, 80%, or 90% of the level of endogenous stem cell proliferation in an age-matched healthy control. The level of proliferation may be assessed by functional MRI or other imagining means. Additionally, endogenous stem cell proliferation may be assessed by pathophysiological characteristics. An abnormally high level of IL-17 in an individual may be determined by measuring levels of IL-17 in the plasma, peripheral blood, and/or cerebral spinal fluid of the individual. In some embodiments, a measured IL-17 level in an individual that is approximately 10%, 20%, 30%, 40%, 50%, 60%, 70%, 80%, 90%, or 100% higher or more than what is found in an age-matched healthy control is an abnormally high level of IL-17 in the individual. In some embodiments, the levels of IL-17 are assessed in cells, including mononuclear cells, CD4 cells, and/or Th17 cells, from peripheral blood subsequent to stimulation.

An abnormally low level of IL-10 in an individual may be determined by measuring levels of IL-10 in the plasma, peripheral blood, and/or cerebral spinal fluid of the individual. In some embodiments, a measured IL-10 level in an individual that is approximately <10%, 10%, 20%, 30%, 40%, 50%, 60%, 70%, 80%, or 90% of the level that is found in an age-matched healthy control is an abnormally low level of IL-10 in the individual. In some embodiments, the levels of IL-10 are assessed in cells, including mononuclear cells, CD4 cells, and/or Th17 cells, from peripheral blood subsequent to stimulation.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Allogeneic,” as used herein, refers to cells of the same species that differ genetically from cells of a host.

“Autologous,” as used herein, refers to cells derived from the same subject.

The term “engraft” as used herein refers to the process of stem cell incorporation into a tissue of interest in vivo through contact with existing cells of the tissue.

“Xenogeneic,” as used herein, refers to cells derived from, originating in, or being a member of another species.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Carrier” or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

As used herein, the term “expresses”, when referring to a gene, nucleic acid, protein, cell marker, or the like, means that expression of the gene, nucleic acid, protein, cell marker can be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). For gene expression, a gene is said to be expressed if the presence of its mRNA can be detected using standard methods. For example, a gene can be said to be expressed by a cell if the mRNA transcribed from the gene can be detected on a standard agarose gel following standard PCR protocols.

The term “culture expanded population” means a population of cells whose numbers have been increased by cell division in vitro. This term may apply to stem cell populations and non-stem cell populations alike.

The term “individual” generally refers to an individual in need of a therapy. The individual can be a mammal, such as a human, dog, cat, horse, pig or rodent. The individual can be a patient, e.g., have or be suspected of having or at risk for having a disease or medical condition related to bone. For individuals having or suspected of having a medical condition directly or indirectly associated with bone, the medical condition may be of one or more types. The individual may have a disease or be suspected of having the disease. The individual may be asymptomatic. The individual may be of any gender. The individual may be of a certain age, such as at least 20, 25, 30, 35, 40, 45, 50, 55, 60, 65, 70, 75, 80, 85, 90, 95, or 100 or more.

The term “passaging” refers to the process of transferring a portion of cells from one culture vessel into a new culture vessel.

The term “cryopreserve” refers to preserving cells for long term storage in a cryoprotectant at low temperature.

The term “master cell bank” refers to a collection of cryopreserved cells. Such a cell bank may comprise stem cells, non-stem cells, and/or a mixture of stem cells and non-stem cells.

The terms “ToleroChyme” or “Tolerochymal stem cell” as used herein refer to stem cells with the ability to modulate the immune system to block or prevent conditions including but not limited to colitis. In some embodiments, ToleroChyme cells have the ability to modulate the immune system. In some embodiments, ToleroChyme cells are produced from umbilical cords. In some embodiments, ToleroChyme cells can be used in a “universal donor” fashion, meaning that cells from one donor can be used to treat multiple recipients. In some embodiments, ToleroChyme cells can be administered to the colon wall. In some embodiments, Tolerochyme may be mesenchymal stem cells selected for CD56 and subsequently activated with one or more inflammatory stimuli.

Certain embodiments encompassed herein concern the administration of exosomes to an individual. The individual may have UC. The individual may have one or more abnormalities associated with UC, including any abnormality encompassed herein. In some embodiments, an individual is administered an effective amount of a population of exosomes. The exosomes may be administered at a concentration and frequency sufficient to correct or ameliorate one or more abnormalities associated with MS. In some embodiments, the concentration and/or frequency of administration is adjusted based on the response of the individual to the administration of exosomes.

In some embodiments the disclosure teaches the use of perinatal tissue derived exosomes for stimulating generation of myeloid derived suppressor cells. Myeloid-derived suppressor cells (MDSCs) are a heterogeneous population of cells that arise from myeloid progenitor cells and are known to suppress immune responses. MDSCs have been shown to play a critical role in dampening immune responses in a variety of conditions such as infections, cancer, and autoimmunity. There are several mechanisms by which MDSCs can suppress immune responses. One mechanism is through the production of reactive oxygen species (ROS) and nitric oxide (NO), which can inhibit T cell proliferation and function. MDSCs can also produce arginase, which competes with T cells for the amino acid arginine, thereby reducing T cell activation and proliferation. In addition, MDSCs can express surface molecules such as PD-L1 and CTLA-4, which interact with T cell receptors and inhibit T cell activation. MDSCs can also produce cytokines such as IL-10 and TGF-beta, which can suppress immune responses by inhibiting T cell activation and promoting the generation of regulatory T cells. Overall, the suppressive function of MDSCs is thought to be mediated through a combination of these mechanisms, which act to limit the activation and proliferation of immune cells and promote the generation of regulatory T cells. In one embodiment, stimulation of MDSC is accomplished by administration of perinatal tissue derived exosomes and Tysabri. Tysabri (natalizumab) is a medication used to treat multiple sclerosis (MS) and Crohn's disease. Tysabri works by targeting specific immune cells in the body called alpha-4 integrins, which are involved in inflammation and immune cell migration.

In some embodiments of the disclosure, administration of low doses of IL-2 in the form of aldesleukin every day at concentrations of 0.3×10⁶ to 3.0×10⁶ IU IL-2 per square meter of body surface area for 8 weeks is provided together with administration of perinatal tissue derived cells or exosomes originated from said cells, or in other embodiments repetitive 5-day courses of 1.0×10⁶ to 3.0×10⁶ IU IL-2 together with exosomes. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes.

In some embodiments of the disclosure, administration of estrogen is performed in order to assist in the expansion of Treg cells. The utilization of estrogen to advance expansion of Treg cells has previously been demonstrated and is incorporated by reference. For example, it is known that CD4(+) CD25(+) regulatory T cells are crucial to the maintenance of tolerance in normal individuals. In augmenting FoxP3 expression in vitro and in vivo. Investigators showed that treatment of naive mice with estrogen (E2) increased both CD25(+) cell number and FoxP3 expression level. Further, the ability of E2 to protect against autoimmune disease (experimental autoimmune encephalomyelitis) correlated with its ability to up-regulate FoxP3, as both were reduced in estrogen receptor alpha-deficient animals. Finally, E2 treatment and pregnancy induced FoxP3 protein expression to a similar degree, suggesting that high estrogen levels during pregnancy may help to maintain fetal tolerance.

In some embodiments therapeutic exosomes are derived from various types of mesenchymal stem cells. Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. In some embodiments, cells are capable of growth and expansion in chemically-defined growth media with no serum added. In such cases, the cells may require certain growth factors, which can be added to the medium to support and sustain the cells. In some embodiments, factors to be added for growth in serum-free media include one or more of FGF, EGF, IGF, and PDGF. In some embodiments, two, three or all four of the factors are added to serum free or chemically defined media. In some embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 200 doublings prior to reaching a senescent state. Methods for deriving cells capable of doubling to reach 10.sup.14 cells or more are provided. In some embodiments, methods derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. In some embodiments, these cell numbers are produced within 80, 70, or 60 days or less. In some embodiments, cord tissue mesenchymal stem cells are isolated and expanded, and possess one or more markers selected from a group comprising of CD10, CD13, CD44, CD73, CD90, CD141, PDGFr-alpha, or HLA-A, B, C. In addition, the cells do not produce one or more of CD31, CD34, CD45, CD117, CD141, or HLA-DR, DP, DQ.

In order to determine the quality of MSC cultures, flow cytometry is performed on all cultures for surface expression of SH-2, SH-3, SH-4 MSC markers and lack of contaminating CD14− and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG (H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate-controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In some embodiments, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In one embodiment, hematopoietic stem cells are CD34+ cells isolated from the peripheral blood, bone marrow, or umbilical cord blood. Specifically, the hematopoietic stem cells may be derived from the blood system of mammalian animals, including but not limited to human, mouse, rat, and these hematopoietic stem cells may be harvested by isolating from the blood or tissue organs in mammalian animals. Hematopoietic stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and cell proliferation and differentiation,” issued Jan. 1, 2002, which are hereby incorporated by reference in their entireties. In some embodiments, stem cells obtained from the donor are cultured in order to expand the population of stem cells. In some embodiments, stem cells collected from donor sources are not expanded using such techniques. Standard methods can be used to cyropreserve the stem cells.

In some embodiments, where there are risks associated with particular types of stem cells, for example, pluripotent stem cells, said stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of stem cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of stem cells. Certain embodiments incorporate stem cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiment, stem cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In some embodiments, mesenchymal stem cells are cultured with substances capable of maintaining said mesenchymal stem cells in an immature state, and/or maintaining high expression of genes/mitochondria necessary to prevent, inhibit, and/or reverse Leigh Syndrome. In some embodiments, substances are selected from the group consisting of reversin, cord blood serum, lithium, a GSK-3 inhibitor, resveratrol, pterostilbene, selenium, a selenium-containing compound, EGCG ((−)-epigallocatechin-3-gallate), valproic acid and salts of valproic acid, in particular sodium valproate. In some embodiments, a concentration of reversin from 0.5 to 10 .mu.M, preferably of 1 .mu.M is added to the mesenchymal stem cell culture. In some embodiments, resveratrol is used in a concentration of 10 to 100 .mu.M. In some embodiments resveratrol is used in a concentration of 50 .mu.M. In some embodiments, selenium or a selenium containing compound is used in a concentration from 0.05 to 0.5 .mu.M. In some embodiments, selenium or a selenium containing compound is used in a concentration of 0.1 .mu.M. In some embodiments, cord blood serum is added at a concentration of 0.1%-20% volume to the volume of tissue culture media.

In some embodiments, EGCG is used in a concentration from 0.001 to 0.1 .mu.M. In some embodiments, EGCG is used in a concentration of 0.01 .mu.M. In some embodiments, valproic acid or sodium valproate is used in a concentration of 5 .mu.M.

In some embodiments, mesenchymal stem cells are retrodifferentiated to possess higher expression of regenerative genes. Said retrodifferentiation may be achieved by cytoplasmic transfer, transfection of cytoplasm, or cell fusion with a stem cell possessing a higher level of immaturity, said stem cells including pluripotent stem cells. In such culture/coculture procedures, the cell culture medium comprises, optionally in combination with one or more of the substances specified above, at least one transient proteolysis inhibitor. The use of at least one proteolysis inhibitor in the cell culture medium of the present disclosure increases the time the reprogramming proteins derived from the mRNA or any endogenous genes will be present in the cells and thus facilitates in an even more improved way the reprogramming by the transfected mRNA derived factors. The present disclosure uses in a particular embodiment a transient proteolysis inhibitor a protease inhibitor, a proteasome inhibitor and/or a lysosome inhibitor. In some embodiments, the proteosome inhibitor is selected from the group consisting of MG132, TMC-95A, TS-341 and MG262. In some embodiments, the protease inhibitor is selected from the group consisting of aprotinin, G-64 and leupeptine-hemisulfat. In some embodiments, the lysosomal inhibitor is ammonium chloride.

In some embodiments, a cell culture medium comprises at least one transient inhibitor of mRNA degradation. The use of a transient inhibitor of mRNA degradation increases the half-life of the reprogramming factors as well. In some embodiments, a condition suitable to allow translation of the transfected reprogramming mRNA molecules in the cells is an oxygen content in the cell culture medium from 0.5 to 21%. More particular, and without wishing to be bound to the theory, oxygen is used to further induce or increase Oct4 by triggering Oct4 via Hif1a, in these situations, concentrations of oxygen lower than atmospheric concentration are used, and can be ranging from 0.1% to 10%. In some embodiments, conditions that are suitable to support reprogramming of the cells by the mRNA molecules in the cells are selected; more particularly, these conditions require a temperature from 30 to 38. degree. C. In some embodiments, the conditions require a temperature from 31 to 37. degree. C. In some embodiments, the conditions require a temperature from 32 to 36. degree. C.

In some embodiments, the glucose content of the medium is below 4.6 g/l. In some embodiments, the glucose content of the medium is below 4.5 g/l. In some embodiments, the glucose content of the medium is below 4 g/l. In some embodiments, the glucose content of the medium is below 3 g/l. In some embodiments, the glucose content of the medium is below 2 g/I. In some embodiments, the glucose content of the medium is 1 g/l.

In some embodiments, the media comprises DMEM media containing 1 g/l glucose and is commercially available as “DMEM low glucose” from companies such as PAA, Omega Scientific, Perbio and Biosera. More particularly, and without wishing to be bound to the theory, high glucose conditions adversely support aging of cells (methylation, epigenetics) in vitro which may render the reprogramming difficult. In some embodiments, the cell culture medium contains glucose in a concentration from 0.1 g/l to 4.6 g/l. In some embodiments, the cell culture medium contains glucose in a concentration from 0.5 g/l to 4.5 g/l. In some embodiments, the cell culture medium contains glucose in a concentration from 1 g/l to 4 g/l.

Various sources of mesenchymal stem cells may be used, depending on tissue and age. Examples of somatic cells which may be used as the donor cell for transdifferentiation include any cell type that is desired for cell therapies are cells relevant to pathology of Leigh Syndrome. The current disclosure further provides dedifferentiation of target cells using total RNA or mRNA. The mRNA or total RNA used to effect dedifferentiation is preferably isolated from cells that are either pluripotent or which are capable of turning into pluripotent cells (oocyte). Examples thereof include by way of example Ntera cells, human or other ES cells, primordial germ cells, and blastocysts. Alternatively, the RNA used to effect dedifferentiation may comprise mRNA encoding specific transcription factors. The total RNA or mRNA's may be delivered into target cells by different methods including, e.g., electroporation, liposomes, and mRNA injection. Target cells into which RNA's are introduced and which are to be dedifferentiated according to the disclosure are cultured in a medium containing one or more constituents that facilitates transformation of cell phenotype. These constituents include by way of example epigenetic modifiers such as DNA demethylating agents, HDAC inhibitors, histone modifiers; and cell cycle manipulation and pluripotent or tissue specific promoting agents such as helper cells which promote growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, procarbazine, et al. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Preferred examples of histone modifiers include PARP, the human enhancer of zeste, valproic acid, and trichostatine. Particular constituents that the inventors utilize in a preferred media in order to facilitate RNA transformation and dedifferentiation of the RNA comprising target cells into pluripotent cells include trichostatine, valproic acid, zebularine and 5-aza. Target cells into which RNA is introduced are cultured for a sufficient time in media that promotes RNA transformation until dedifferentiated cells (pluripotent) cells are obtained.

In some instances, this methodology may be combined with other methods and treatments involved in the epigenetic status of the recipient or target cell such as the exposure to DNA and histone demethylating agents, histone deacetylase inhibitors, and/or histone modifiers. This disclosure therefore describes a method of changing the fate or phenotype of cells. By using epigenetic modifications, the subject methods can dedifferentiate or transdifferentiate cells. The compositions and methods of this disclosure are aimed to solve the problem of immuno-rejection which is evident when incompatible cells/tissues are used for transplantation. Cells from one patient can be transformed into a different type of cell allowing for the derivation of cells needed for the treatment of a particular disease the patient is suffering from. One of the types of cells that can be produced by the compositions and methods of this disclosure is pluripotent stem cells. The compositions and methods of this disclosure offer an opportunity to the research community to study the mechanisms involved in cell differentiation and disease progression.

In addition, the recipient cells may be cultured under different conditions that enhance reprogramming efficiency such as co-culture of the RNA transfected cells with other cell types, conditioned medias, and by the supplementation of the culture medium with other biological agents such as growth factors, hormones, vitamins, etc. which enhance growth and maintenance of the cultured cells. In some embodiments, mesenchymal stem cells are treated with one or more “Inhibitor(s) of DNA methylation”. This term refers to an agent that can inhibit DNA methylation. DNA methylation inhibitors have demonstrated the ability to restore suppressed gene expression. Suitable agents for inhibiting DNA methylation include, but are not limited to 5-azacytidine, 5-aza-2-deoxycytidine, 1-.beta.-D-arabinofuranosil-5-azacytosine, and dihydro-5-azacytidine, and zebularine (ZEB), BIX (histone lysine methyltransferase inhibitor), and RG108. Concentration of DNA methylation inhibitors, as well as duration of exposure, is dependent on ability to induce expansion of plasticity.

Also disclosed herein for use in culture of mesenchymal stem cells are inhibitors of deacetylation. This term refers to an agent that prevents the removal of the acetyl groups from the lysine residues of histones that would lead to the formation of condensed and transcriptionally silenced chromatin. Histone deacetylase inhibitors fall into several groups, including: (1) hydroxamic acids such as trichostatin (A), (2) cyclic tetrapeptides, (3) benzamides, (4) electrophilic ketones, and (5) aliphatic acid group of compounds such as phenylbutyrate and valporic acid. Suitable agents to inhibit histone deacetylation include, but are not limited to, valporic acid, phenylbutyrate and Trichostatin A (TSA). One example, in the area of mesenchymal stem cells, of valproic acid enhancing pluripotency and therapeutic properties is provided in a study which showed that culture of cells with valproic acid enhanced immune regulatory and metabolic properties of mesenchymal stem cells. The culture systems described, as well as means of assessment, are provided to allow one of skill in the art to have a starting point for the practice of the current disclosure. Without being bound to theory, valproic acid in the context of the current disclosure may be useful to increasing in vitro proliferation of dedifferentiated mesenchymal stem cells while preventing senescence associated stress. For example, Zhai et al showed that in an in vitro pre-mature senescence model, valproic acid treatment increased cell proliferation and inhibited apoptosis through the suppression of the p16/p21 pathway. In addition, valproic acid also inhibited the G2/M phase blockage derived from the senescence stress.

Additional, non-limiting embodiments of the present disclosure are provided in the following numbered arrangements.

1. A method of preventing and/or treating colitis comprising administration of a population of mesenchymal stem cells possessing enhanced ability to induce generation of T regulatory cells as compared to conventional mesenchymal stem cells and optionally administered separately or together a cell-free means or composition possessing therapeutic potential.

2. The method of Arrangement 1, wherein said colitis is mediated by an autoimmune response in patient suffering from said colitis.

3. The method of Arrangement 2, wherein said autoimmune response is associated with activation of Th17 cells.

4. The method of Arrangement 3, wherein said Th17 cells are associated with reduced generation of T regulatory cells.

5. The method of Arrangement 3, wherein said Th17 cells are associated with reduced generation of T regulatory 10 (Tr10) cells.

6. The method of Arrangement 1, wherein said T regulatory cells express FoxP3.

7. The method of Arrangement 1, wherein said T regulatory cells express Fas ligand.

8. The method of Arrangement 1, wherein said T regulatory cells express granzyme B.

9. The method of Arrangement 1, wherein said T regulatory cells express perforin.

10. The method of Arrangement 1, wherein said T regulatory cells express membrane bound TGF-beta.

11. The method of Arrangement 1, wherein said T regulatory cells suppress dendritic cell maturation.

12. The method of Arrangement 11, wherein said dendritic cell maturation is associated with upregulation of interleukin-12 production.

13. The method of Arrangement 11, wherein said dendritic cell maturation is associated with upregulation of interleukin-1 beta production.

14. The method of Arrangement 11, wherein said dendritic cell maturation is associated with upregulation of interleukin-2 production.

15. The method of Arrangement 11, wherein said dendritic cell maturation is associated with upregulation of interleukin-6 production.

16. The method of Arrangement 11, wherein said dendritic cell maturation is associated with upregulation of TNF-alpha production.

17. The method of Arrangement 11, wherein said dendritic cell maturation is associated with upregulation of lymphotoxin production.

18. The method of Arrangement 11, wherein said dendritic cell maturation is associated with upregulation of TRAIL production.

19. The method of Arrangement 11, wherein said dendritic cell maturation is associated with upregulation of TWEAK production.

20. The method of Arrangement 11, wherein said dendritic cell maturation is associated with down regulation of interleukin-10 production.

21. The method of Arrangement 11, wherein said dendritic cell maturation is associated with down regulation of interleukin-12 p40 homodimer production.

22. The method of Arrangement 11, wherein said dendritic cell maturation is associated with down regulation of soluble HLA-G production.

23. The method of Arrangement 11, wherein said dendritic cell maturation is associated with down regulation of soluble TNF-alpha receptor p55 production.

24. The method of Arrangement 11, wherein said dendritic cell maturation is associated with down regulation of soluble TNF-alpha receptor p75 production.

25. The method of Arrangement 11, wherein said dendritic cell maturation is associated with down regulation of soluble TNF-alpha receptor p55: p75 heterodimer production.

26. The method of Arrangement 11, wherein said dendritic cell maturation is associated with upregulation of CD5 expression.

27. The method of Arrangement 11, wherein said dendritic cell maturation is associated with upregulation of CD40 expression.

28. The method of Arrangement 11, wherein said dendritic cell maturation is associated with upregulation of CD80 expression.

29. The method of Arrangement 11, wherein said dendritic cell maturation is associated with upregulation of CD86 expression.

30. The method of Arrangement 11, wherein said dendritic cell maturation is associated with upregulation of HLA II expression.

31. The method of Arrangement 1, wherein said mesenchymal stem cells possessing enhanced ability to induce generation of T regulatory cells as compared to conventional mesenchymal stem cells are autologous to the patient being treated.

32. The method of Arrangement 1, wherein said mesenchymal stem cells possessing enhanced ability to induce generation of T regulatory cells as compared to conventional mesenchymal stem cells are allogeneic to the patient being treated.

33. The method of Arrangement 1, wherein said mesenchymal stem cells possessing enhanced ability to induce generation of T regulatory cells as compared to conventional mesenchymal stem cells are xenogeneic to the patient being treated.

34. The method of Arrangement 1, wherein said mesenchymal stem cells possessing enhanced ability to induce generation of T regulatory cells as compared to conventional mesenchymal stem cells are derived from perinatal tissue.

35. The method of Arrangement 34, wherein said perinatal tissue derived mesenchymal stem cells are obtained from Wharton's Jelly.

36. The method of Arrangement 34, wherein said perinatal tissue derived mesenchymal stem cells are obtained from subepithelial cord tissue.

37. The method of Arrangement 35, wherein said Wharton's Jelly mesenchymal stem cell is plastic adherent.

38. The method of Arrangement 35, wherein said Wharton's Jelly mesenchymal stem cell is collagen II adherent.

39. The method of Arrangement 35, wherein said Wharton's Jelly mesenchymal stem cell is fibronectin adherent.

40. The method of Arrangement 35, wherein said Wharton's Jelly mesenchymal stem cell is adherent to hyaluronic acid.

41. The method of Arrangement 35, wherein said Wharton's Jelly mesenchymal stem cell is aggrecan adherent.

42. The method of Arrangement 35, wherein said Wharton's Jelly mesenchymal stem cell is select for enhanced expression of CD56 as compared to other mesenchymal stem cells from Wharton's Jelly.

43. The method of Arrangement 42, wherein said cells with enhanced expression of CD56 are selected after culture of Wharton's Jelly derived mesenchymal stem cells for at least 24 hours with interferon gamma.

44. The method of Arrangement 43, wherein said concentration of interferon gamma is sufficient to enhance expression of HLA II at least 50% as compared to baseline.

45. The method of Arrangement 43, wherein said concentration of interferon gamma is sufficient to enhance expression of HLA II at least 100% as compared to baseline.

46. The method of Arrangement 43, wherein said concentration of interferon gamma is sufficient to enhance expression of HLA II at least 200% as compared to baseline.

47. The method of Arrangement 42, wherein said cells with enhanced expression of CD56 are selected after culture of Wharton's Jelly derived mesenchymal stem cells for at least 48 hours with interferon gamma.

48. The method of Arrangement 47, wherein said concentration of interferon gamma is sufficient to enhance expression of HLA II at least 50% as compared to baseline.

49. The method of Arrangement 47, wherein said concentration of interferon gamma is sufficient to enhance expression of HLA II at least 100% as compared to baseline.

50. The method of Arrangement 47, wherein said concentration of interferon gamma is sufficient to enhance expression of HLA II at least 200% as compared to baseline.

51. The method of Arrangement 42, wherein said cells with enhanced expression of CD56 are selected after culture of Wharton's Jelly derived mesenchymal stem cells for at least 72 hours with interferon gamma.

52. The method of Arrangement 51, wherein said concentration of interferon gamma is sufficient to enhance expression of HLA II at least 50% as compared to baseline.

53. The method of Arrangement 51, wherein said concentration of interferon gamma is sufficient to enhance expression of HLA II at least 100% as compared to baseline.

54. The method of Arrangement 51, wherein said concentration of interferon gamma is sufficient to enhance expression of HLA II at least 200% as compared to baseline.

55. The method of Arrangement 42, wherein said cells with enhanced expression of CD73 are selected after culture of Wharton's Jelly derived mesenchymal stem cells for at least 24 hours with interferon gamma.

56. The method of Arrangement 55, wherein said concentration of interferon gamma is sufficient to enhance expression of HLA II at least 50% as compared to baseline.

57. The method of Arrangement 55, wherein said concentration of interferon gamma is sufficient to enhance expression of HLA II at least 100% as compared to baseline.

58. The method of Arrangement 55, wherein said concentration of interferon gamma is sufficient to enhance expression of HLA II at least 200% as compared to baseline.

59. The method of Arrangement 42, wherein said cells with enhanced expression of CD73 are selected after culture of Wharton's Jelly derived mesenchymal stem cells for at least 48 hours with interferon gamma.

60. The method of Arrangement 55, wherein said concentration of interferon gamma is sufficient to enhance expression of HLA II at least 50% as compared to baseline.

61. The method of Arrangement 55, wherein said concentration of interferon gamma is sufficient to enhance expression of HLA II at least 100% as compared to baseline.

62. The method of Arrangement 55, wherein said concentration of interferon gamma is sufficient to enhance expression of HLA II at least 200% as compared to baseline.

63. The method of Arrangement 42, wherein said cells with enhanced expression of CD73 are selected after culture of Wharton's Jelly derived mesenchymal stem cells for at least 72 hours with interferon gamma.

64. The method of Arrangement 55, wherein said concentration of interferon gamma is sufficient to enhance expression of HLA II at least 50% as compared to baseline.

65. The method of Arrangement 55, wherein said concentration of interferon gamma is sufficient to enhance expression of HLA II at least 100% as compared to baseline.

66. The method of Arrangement 55, wherein said concentration of interferon gamma is sufficient to enhance expression of HLA II at least 200% as compared to baseline.

67. The Arrangement of claim 35, wherein said Wharton's Jelly derived mesenchymal stem cell is cultured under conditions to enhance expression of HLA-G.

68. The Arrangement of claim 67, wherein said culture conditions to increase expression of HLA-G are exposure to interleukin-10 at a concentration and duration suppress interferon induced expression of HLA II by over 25% as compared to baseline expression of HLA II induced by interferon gamma.

69. The Arrangement of claim 68, wherein said interferon gamma is administered at a concentration of 1-10,000 IU/million mesenchymal stem cells.

70. The Arrangement of claim 68, wherein said interferon gamma is administered at a concentration of 1-1,000 IU/million mesenchymal stem cells.

71. The Arrangement of claim 68, wherein said interferon gamma is administered at a concentration of 10-100 IU/million mesenchymal stem cells.

72. The Arrangement of claim 67, wherein said culture conditions to increase expression of HLA-G are exposure to interleukin-10 at a concentration and duration suppress interferon induced expression of HLA II by over 50% as compared to baseline expression of HLA II induced by interferon gamma.

73. The Arrangement of claim 67, wherein said culture conditions to increase expression of HLA-G are exposure to interleukin-10 at a concentration and duration suppress interferon induced expression of HLA II by over 75% as compared to baseline expression of HLA II induced by interferon gamma.

74. The Arrangement of claim 67, wherein said culture conditions to increase expression of HLA-G are exposure to interleukin-4 at a concentration and duration suppress interferon induced expression of HLA II by over 25% as compared to baseline expression of HLA II induced by interferon gamma.

75. The Arrangement of claim 67, wherein said culture conditions to increase expression of HLA-G are exposure to interleukin-4 at a concentration and duration suppress interferon induced expression of HLA II by over 50% as compared to baseline expression of HLA II induced by interferon gamma.

76. The Arrangement of claim 67, wherein said culture conditions to increase expression of HLA-G are exposure to interleukin-4 at a concentration and duration suppress interferon induced expression of HLA II by over 75% as compared to baseline expression of HLA II induced by interferon gamma.

77. The Arrangement of claim 67, wherein said culture conditions to increase expression of HLA-G are exposure to interleukin-13 at a concentration and duration suppress interferon induced expression of HLA II by over 25% as compared to baseline expression of HLA II induced by interferon gamma.

78. The Arrangement of claim 67, wherein said culture conditions to increase expression of HLA-G are exposure to interleukin-13 at a concentration and duration suppress interferon induced expression of HLA II by over 50% as compared to baseline expression of HLA II induced by interferon gamma.

79. The Arrangement of claim 67, wherein said culture conditions to increase expression of HLA-G are exposure to interleukin-13 at a concentration and duration suppress interferon induced expression of HLA II by over 75% as compared to baseline expression of HLA II induced by interferon gamma.

80. The Arrangement of claim 67, wherein said culture conditions to increase expression of HLA-G are exposure to interleukin-20 at a concentration and duration suppress interferon induced expression of HLA II by over 25% as compared to baseline expression of HLA II induced by interferon gamma.

81. The Arrangement of claim 67, wherein said culture conditions to increase expression of HLA-G are exposure to interleukin-20 at a concentration and duration suppress interferon induced expression of HLA II by over 50% as compared to baseline expression of HLA II induced by interferon gamma.

82. The Arrangement of claim 67, wherein said culture conditions to increase expression of HLA-G are exposure to interleukin-20 at a concentration and duration suppress interferon induced expression of HLA II by over 75% as compared to baseline expression of HLA II induced by interferon gamma.

83. The Arrangement of claim 67, wherein said culture conditions to increase expression of HLA-G are exposure to interleukin-22 at a concentration and duration suppress interferon induced expression of HLA II by over 25% as compared to baseline expression of HLA II induced by interferon gamma.

84. The Arrangement of claim 67, wherein said culture conditions to increase expression of HLA-G are exposure to interleukin-22 at a concentration and duration suppress interferon induced expression of HLA II by over 50% as compared to baseline expression of HLA II induced by interferon gamma.

85. The Arrangement of claim 67, wherein said culture conditions to increase expression of HLA-G are exposure to interleukin-22 at a concentration and duration suppress interferon induced expression of HLA II by over 75% as compared to baseline expression of HLA II induced by interferon gamma.

86. The Arrangement of claim 67, wherein said culture conditions to increase expression of HLA-G are exposure to interleukin-35 at a concentration and duration suppress interferon induced expression of HLA II by over 25% as compared to baseline expression of HLA II induced by interferon gamma.

87. The Arrangement of claim 67, wherein said culture conditions to increase expression of HLA-G are exposure to interleukin-35 at a concentration and duration suppress interferon induced expression of HLA II by over 50% as compared to baseline expression of HLA II induced by interferon gamma.

88. The Arrangement of claim 67, wherein said culture conditions to increase expression of HLA-G are exposure to interleukin-35 at a concentration and duration suppress interferon induced expression of HLA II by over 75% as compared to baseline expression of HLA II induced by interferon gamma.

89. The Arrangement of claim 67, wherein said culture conditions to increase expression of HLA-G are exposure to interleukin-37 at a concentration and duration suppress interferon induced expression of HLA II by over 25% as compared to baseline expression of HLA II induced by interferon gamma.

90. The Arrangement of claim 67, wherein said culture conditions to increase expression of HLA-G are exposure to interleukin-37 at a concentration and duration suppress interferon induced expression of HLA II by over 50% as compared to baseline expression of HLA II induced by interferon gamma.

91. The Arrangement of claim 67, wherein said culture conditions to increase expression of HLA-G are exposure to interleukin-37 at a concentration and duration suppress interferon induced expression of HLA II by over 75% as compared to baseline expression of HLA II induced by interferon gamma.

92. The Arrangement of claim 67, wherein said culture conditions to increase expression of HLA-G are exposure to interleukin-38 at a concentration and duration suppress interferon induced expression of HLA II by over 25% as compared to baseline expression of HLA II induced by interferon gamma.

93. The Arrangement of claim 67, wherein said culture conditions to increase expression of HLA-G are exposure to interleukin-38 at a concentration and duration suppress interferon induced expression of HLA II by over 50% as compared to baseline expression of HLA II induced by interferon gamma.

94. The Arrangement of claim 67, wherein said culture conditions to increase expression of HLA-G are exposure to interleukin-38 at a concentration and duration suppress interferon induced expression of HLA II by over 75% as compared to baseline expression of HLA II induced by interferon gamma.

95. The Arrangement of claim 67, wherein said culture conditions to increase expression of HLA-G are exposure to VEGF at a concentration and duration suppress interferon induced expression of HLA II by over 25% as compared to baseline expression of HLA II induced by interferon gamma.

96. The Arrangement of claim 67, wherein said culture conditions to increase expression of HLA-G are exposure to VEGF at a concentration and duration suppress interferon induced expression of HLA II by over 50% as compared to baseline expression of HLA II induced by interferon gamma.

97. The Arrangement of claim 67, wherein said culture conditions to increase expression of HLA-G are exposure to VEGF at a concentration and duration suppress interferon induced expression of HLA II by over 75% as compared to baseline expression of HLA II induced by interferon gamma.

98. The Arrangement of claim 67, wherein said culture conditions to increase expression of HLA-G are exposure to EGF at a concentration and duration suppress interferon induced expression of HLA II by over 25% as compared to baseline expression of HLA II induced by interferon gamma.

99. The Arrangement of claim 67, wherein said culture conditions to increase expression of HLA-G are exposure to EGF at a concentration and duration suppress interferon induced expression of HLA II by over 50% as compared to baseline expression of HLA II induced by interferon gamma.

100. The Arrangement of claim 67, wherein said culture conditions to increase expression of HLA-G are exposure to EGF at a concentration and duration suppress interferon induced expression of HLA II by over 75% as compared to baseline expression of HLA II induced by interferon gamma.

101. The Arrangement of claim 67, wherein said culture conditions to increase expression of HLA-G are exposure to FGF-1 at a concentration and duration suppress interferon induced expression of HLA II by over 25% as compared to baseline expression of HLA II induced by interferon gamma.

102. The Arrangement of claim 67, wherein said culture conditions to increase expression of HLA-G are exposure to FGF-1 at a concentration and duration suppress interferon induced expression of HLA II by over 50% as compared to baseline expression of HLA II induced by interferon gamma.

103. The Arrangement of claim 67, wherein said culture conditions to increase expression of HLA-G are exposure to FGF-1 at a concentration and duration suppress interferon induced expression of HLA II by over 75% as compared to baseline expression of HLA II induced by interferon gamma.

104. The Arrangement of claim 67, wherein said culture conditions to increase expression of HLA-G are exposure to FGF-2 at a concentration and duration suppress interferon induced expression of HLA II by over 25% as compared to baseline expression of HLA II induced by interferon gamma.

105. The Arrangement of claim 67, wherein said culture conditions to increase expression of HLA-G are exposure to FGF-2 at a concentration and duration suppress interferon induced expression of HLA II by over 50% as compared to baseline expression of HLA II induced by interferon gamma.

106. The Arrangement of claim 67, wherein said culture conditions to increase expression of HLA-G are exposure to FGF-2 at a concentration and duration suppress interferon induced expression of HLA II by over 75% as compared to baseline expression of HLA II induced by interferon gamma.

107. The Arrangement of claim 67, wherein said culture conditions to increase expression of HLA-G are exposure to FGF-5 at a concentration and duration suppress interferon induced expression of HLA II by over 25% as compared to baseline expression of HLA II induced by interferon gamma.

108. The Arrangement of claim 67, wherein said culture conditions to increase expression of HLA-G are exposure to FGF-5 at a concentration and duration suppress interferon induced expression of HLA II by over 50% as compared to baseline expression of HLA II induced by interferon gamma.

109. The Arrangement of claim 67, wherein said culture conditions to increase expression of HLA-G are exposure to FGF-5 at a concentration and duration suppress interferon induced expression of HLA II by over 75% as compared to baseline expression of HLA II induced by interferon gamma.

110. The Arrangement of claim 67, wherein said culture conditions to increase expression of HLA-G are exposure to HGF at a concentration and duration suppress interferon induced expression of HLA II by over 25% as compared to baseline expression of HLA II induced by interferon gamma.

111. The Arrangement of claim 67, wherein said culture conditions to increase expression of HLA-G are exposure to HGF at a concentration and duration suppress interferon induced expression of HLA II by over 50% as compared to baseline expression of HLA II induced by interferon gamma.

112. The Arrangement of claim 67, wherein said culture conditions to increase expression of HLA-G are exposure to HGF at a concentration and duration suppress interferon induced expression of HLA II by over 75% as compared to baseline expression of HLA II induced by interferon gamma.

113. The Arrangement of claim 67, wherein said culture conditions to increase expression of HLA-G are exposure to interferon beta at a concentration and duration suppress interferon induced expression of HLA II by over 25% as compared to baseline expression of HLA II induced by interferon gamma.

114. The Arrangement of claim 67, wherein said culture conditions to increase expression of HLA-G are exposure to interferon beta at a concentration and duration suppress interferon induced expression of HLA II by over 50% as compared to baseline expression of HLA II induced by interferon gamma.

115. The Arrangement of claim 67, wherein said culture conditions to increase expression of HLA-G are exposure to interferon beta at a concentration and duration suppress interferon induced expression of HLA II by over 75% as compared to baseline expression of HLA II induced by interferon gamma.

116. The Arrangement of claim 67, wherein said culture conditions to increase expression of HLA-G are exposure to angiopoietin at a concentration and duration suppress interferon induced expression of HLA II by over 25% as compared to baseline expression of HLA II induced by interferon gamma.

117. The Arrangement of claim 67, wherein said culture conditions to increase expression of HLA-G are exposure to angiopoietin at a concentration and duration suppress interferon induced expression of HLA II by over 50% as compared to baseline expression of HLA II induced by interferon gamma.

118. The Arrangement of claim 67, wherein said culture conditions to increase expression of HLA-G are exposure to angiopoietin at a concentration and duration suppress interferon induced expression of HLA II by over 75% as compared to baseline expression of HLA II induced by interferon gamma.

119. The Arrangement of claim 67, wherein said culture conditions to increase expression of HLA-G are exposure to placental growth factor at a concentration and duration suppress interferon induced expression of HLA II by over 25% as compared to baseline expression of HLA II induced by interferon gamma.

120. The Arrangement of claim 67, wherein said culture conditions to increase expression of HLA-G are exposure to placental growth factor at a concentration and duration suppress interferon induced expression of HLA II by over 50% as compared to baseline expression of HLA II induced by interferon gamma.

121. The Arrangement of claim 67, wherein said culture conditions to increase expression of HLA-G are exposure to placental growth factor at a concentration and duration suppress interferon induced expression of HLA II by over 75% as compared to baseline expression of HLA II induced by interferon gamma.

122. The Arrangement of claim 67, wherein said culture conditions to increase expression of HLA-G are exposure to progesterone induced blocking factor at a concentration and duration suppress interferon induced expression of HLA II by over 25% as compared to baseline expression of HLA II induced by interferon gamma.

123. The Arrangement of claim 67, wherein said culture conditions to increase expression of HLA-G are exposure to progesterone induced blocking factor at a concentration and duration suppress interferon induced expression of HLA II by over 50% as compared to baseline expression of HLA II induced by interferon gamma.

124. The Arrangement of claim 67, wherein said culture conditions to increase expression of HLA-G are exposure to progesterone induced blocking factor at a concentration and duration suppress interferon induced expression of HLA II by over 75% as compared to baseline expression of HLA II induced by interferon gamma.

125. The Arrangement of claim 67, wherein said culture conditions to increase expression of HLA-G are exposure to human chorionic gonadotrophin at a concentration and duration suppress interferon induced expression of HLA II by over 25% as compared to baseline expression of HLA II induced by interferon gamma.

126. The Arrangement of claim 67, wherein said culture conditions to increase expression of HLA-G are exposure to human chorionic gonadotrophin at a concentration and duration suppress interferon induced expression of HLA II by over 50% as compared to baseline expression of HLA II induced by interferon gamma.

127. The Arrangement of claim 67, wherein said culture conditions to increase expression of HLA-G are exposure to human chorionic gonadotrophin at a concentration and duration suppress interferon induced expression of HLA II by over 75% as compared to baseline expression of HLA II induced by interferon gamma.

128. The Arrangement of claim 67, wherein said culture conditions to increase expression of HLA-G are exposure to lithium or a lithium salt at a concentration and duration suppress interferon induced expression of HLA II by over 25% as compared to baseline expression of HLA II induced by interferon gamma.

129. The Arrangement of claim 67, wherein said culture conditions to increase expression of HLA-G are exposure to lithium or a lithium salt at a concentration and duration suppress interferon induced expression of HLA II by over 50% as compared to baseline expression of HLA II induced by interferon gamma.

130. The Arrangement of claim 67, wherein said culture conditions to increase expression of HLA-G are exposure to lithium or a lithium salt at a concentration and duration suppress interferon induced expression of HLA II by over 75% as compared to baseline expression of HLA II induced by interferon gamma.

131. The Arrangement of claim 1, wherein said mesenchymal stem cells do not express CD45.

132. The Arrangement of claim 1, wherein said mesenchymal stem cells do not express CD34.

133. The Arrangement of claim 1, wherein said mesenchymal stem cells do not express HLA-G.

134. The Arrangement of claim 1, wherein said mesenchymal stem cells are capable of suppressing T cell proliferation.

135. The Arrangement of claim 131, wherein said T cell proliferation is induced by stimulation of said T cells with one or more mitogens.

136. The Arrangement of claim 132, wherein said mitogen is a lectin.

137. The Arrangement of claim 132, wherein said mitogen is phytohemagglutinin.

138. The Arrangement of claim 132, wherein said mitogen is concanavalin A.

139. The Arrangement of claim 132, wherein said mitogen is one or more antibodies capable of crosslinking the T cell receptor.

140. The Arrangement of claim 132, wherein said mitogen is one or more antibodies capable of crosslinking the T cell receptor and one or more costimulatory receptors.

141. The Arrangement of claim 137, wherein said antibody capable of crosslinking said T cell receptor is an anti-CD3 antibody.

142. The Arrangement of claim 137, wherein said antibody capable of crosslinking said T cell receptor is an anti-TCR antibody.

143. The Arrangement of claim 137, wherein said antibody capable of crosslinking said T cell receptor is an anti-CD52 antibody.

144. The Arrangement of claim 137, wherein said antibody capable of crosslinking a costimulatory molecule is an anti-CD28 antibody.

145. The Arrangement of claim 137, wherein said antibody capable of crosslinking a costimulatory molecule is an anti-CD40 antibody.

146. The Arrangement of claim 137, wherein said antibody capable of crosslinking a costimulatory molecule is an anti-CD5 antibody.

147. The Arrangement of claim 137, wherein said antibody capable of crosslinking a costimulatory molecule is an anti-CD25 antibody.

148. The Arrangement of claim 137, wherein said antibody capable of crosslinking a costimulatory molecule is an anti-ICAM antibody. 149. The Arrangement of claim 1, wherein said cell-free means is a conditioned media.

150. The Arrangement of claim 1, wherein said cell-free means is a protein concentrate.

151. The Arrangement of claim 1, wherein said cell-free means is a concentrated nucleic acid composition.

152. The Arrangement of claim 1, wherein said cell-free means is a concentrated microRNA composition.

153. The Arrangement of claim 1, wherein said cell-free means is composition of concentrated apoptotic bodies.

154. The Arrangement of claim 1, wherein said cell-free means is composition of concentrated exosomes.

155. The Arrangement of claim 1, wherein said cell free means is collected from a pluripotent stem cell.

156. The Arrangement of claim 1, wherein said pluripotent stem cell is an induced pluripotent stem cell.

157. The Arrangement of claim 152, wherein said pluripotent stem cell is a dedifferentiated monocyte.

158. The Arrangement of claim 153, wherein said monocyte is dedifferentiated by exposure to conditioned media from iPSC.

159. The Arrangement of claim 154, wherein said conditioned media is generated by culture of iPSC cells in the form of cellular bodies in a liquid media.

160. The Arrangement of claim 155, wherein said liquid media is Iscove's media. 161. The Arrangement of claim 155, wherein said liquid media is DMEM media.

162. The Arrangement of claim 155, wherein said liquid media is OptiMEM media.

163. The Arrangement of claim 155, wherein said liquid media is EMEM media.

164. The Arrangement of claim 155, wherein said liquid media is RPMI-1640 media.

165. The Arrangement of claim 155, wherein said liquid media is AIM-V media.

166. The Arrangement of claim 155, wherein said cellular body is disassembled once every 2 days.

167. The Arrangement of claim 155, wherein said cellular body is disassembled once every 5 days.

168. The Arrangement of claim 155, wherein said cellular body is disassembled once every 10 days.

169. The Arrangement of claim 155, wherein said cellular body is composed of iPSC cells together with monocytes.

170. The Arrangement of claim 165, wherein said monocytes are first treated with a stressor before admixed with iPSC for generation of conditioned media.

171. The Arrangement of claim 166, wherein said stressor is hypoxia.

172. The Arrangement of claim 166, wherein said stressor is hypertonicity.

173. The Arrangement of claim 166, wherein said stressor is hypotonicity.

174. The Arrangement of claim 166, wherein said stressor is hyperthermia.

175. The Arrangement of claim 166, wherein said stressor is serum starvation.

176. The Arrangement of claim 166, wherein said stressor is mTOR inhibition.

177. The Arrangement of claim 166, wherein said stressor is AMPK activation.

178. The Arrangement of claim 166, wherein said stressor is activation of inflammatory pathways.

179. The Arrangement of claim 174, wherein said inflammatory pathway is MAP kinase.

180. The Arrangement of claim 174, wherein said inflammatory pathway is Janus Activated Kinase.

181. The Arrangement of claim 174, wherein said inflammatory pathway is Signal Transducer and Activator of Transcription-3.

182. The Arrangement of claim 174, wherein said inflammatory pathway is Signal Transducer and Activator of Transcription-5.

183. The Arrangement of claim 174, wherein said inflammatory pathway is Signal Transducer and Activator of Transcription-6.

184. The Arrangement of claim 174, wherein said inflammatory pathway is TLR2.

185. The Arrangement of claim 180, wherein said TLR2 is activated by peptidoglycan.

186. The Arrangement of claim 174, wherein said inflammatory pathway is TLR3.

187. The Arrangement of claim 180, wherein said TLR3 is activated by double stranded RNA.

188. The Arrangement of claim 180, wherein said TLR3 is activated by Poly IC. 189. The Arrangement of claim 180, wherein said TLR3 is activated by Poly LC.

190. The Arrangement of claim 180, wherein said TLR3 is activated by Poly IC: IC.

191. The Arrangement of claim 180, wherein said TLR3 is activated by inactivated influenza virus.

192. The Arrangement of claim 174, wherein said inflammatory pathway is TLR4.

193. The Arrangement of claim 188, wherein said TLR4 activator are neutrophil extracellular traps.

194. The Arrangement of claim 188, wherein said TLR4 activator is beta glucan.

195. The Arrangement of claim 188, wherein said TLR4 activator is HMGB1.

196. The Arrangement of claim 188, wherein said TLR4 activator is lipopolysaccharide.

197. The Arrangement of claim 188, wherein said TLR4 activator is yeast cell wall extract.

198. The Arrangement of claim 188, wherein said TLR4 activator is anti-TLR4 antibody.

199. The Arrangement of claim 154, wherein said iPSC cells are engineered to express an inhibitor of TGF-beta.

200. The Arrangement of claim 195, wherein said inhibitor of TGF-beta is an antibody molecule.

201. The Arrangement of claim 195, wherein said inhibitor of TGF-beta is a cameloid antibody.

202. The Arrangement of claim 195, wherein said inhibitor of TGF-beta is a microbody.

203. The Arrangement of claim 195, wherein said inhibitor of TGF-beta is an aptamer.

204. The Arrangement of claim 195, wherein said inhibitor of TGF-beta is a molecule capable of inducing the process of RNA interference.

205. The Arrangement of claim 200, wherein said molecule capable of inducing RNA interference is a short hairpin RNA.

206. The Arrangement of claim 200, wherein said molecule capable of inducing RNA interference is a short interfering RNA.

207. The Arrangement of claim 200, wherein said molecule capable of inducing RNA interference is a microRNA.

208. The Arrangement of claim 200, wherein said molecule capable of inducing RNA interference is a long noncoding RNA.

209. The Arrangement of claim 1, wherein said cell-free means is extracted or obtained from a regenerative cell, wherein said regenerative cell has been treated with a “promoting agent”, wherein said promoting agent inhibits cellular differentiation while maintaining pluripotency.

210. The Arrangement of claim 205, wherein said promoting agent is GDF-11.

211. The Arrangement of claim 205, wherein said promoting agent is GDF-15.

212. The Arrangement of claim 205, wherein said promoting agent is amniotic fluid.

213. The Arrangement of claim 205, wherein said promoting agent is umbilical cord blood plasma.

214. The Arrangement of claim 205, wherein said promoting agent is BMP2.

215. The Arrangement of claim 205, wherein said promoting agent suppresses NF-kappa B activation.

216. The Arrangement of claim 205, wherein said promoting agent increases NRF2 activation.

217. The Arrangement of claim 205, wherein said promoting agent increases heme-oxygenase-1 activation.

218. The Arrangement of claim 205, wherein said promoting agent increases bcl-2 activation.

219. The Arrangement of claim 205, wherein said promoting agent increases bcl-2XL activation.

220. The Arrangement of claim 205, wherein said promoting agent increases survivin activation.

221. The Arrangement of claim 205, wherein said promoting agent increases livin activation.

222. The Arrangement of claim 205, wherein said promoting agent is an HDAC inhibitor.

223. The Arrangement of claim 218, wherein said HDAC inhibitor is valproic acid.

224. The Arrangement of claim 218, wherein said HDAC inhibitor is trichostatin A.

225. The Arrangement of claim 205, wherein said HDAC inhibitor is sodium phenylbutyrate.

226. The Arrangement of claim 218, wherein said HDAC inhibitor is butyrate.

227. The Arrangement of claim 205, wherein said promoting agent is a GSK-3 inhibitor.

228. The Arrangement of claim 223, wherein said GSK-3 inhibitor is lithium.

229. The Arrangement of claim 205, wherein said promoting agent causes a change in the genotype of the thymic medullary epithelial cell population.

230. The Arrangement of claim 205, wherein at least one promoting agent comprises or consists of an immortalizing oncogene.

231. The Arrangement of claim 205, wherein said promoting agent is PIM1.

232. The Arrangement of claim 226, wherein said promoting agent is SV40 large T antigen.

233. The Arrangement of claim 226, wherein said promoting agent is abl1.

234. The Arrangement of claim 226, wherein said promoting agent is AFF4.

235. The Arrangement of claim 226, wherein said promoting agent is AKT2.

236. The Arrangement of claim 226, wherein said promoting agent is AKL.

237. The Arrangement of claim 226, wherein said promoting agent is AML1.

238. The Arrangement of claim 226, wherein said promoting agent is MTG8.

239. The Arrangement of claim 226, wherein said promoting agent is BCL6.

240. The Arrangement of claim 226, wherein said promoting agent is MCF2.

241. The Arrangement of claim 226, wherein said promoting agent is DCF3.

242. The Arrangement of claim 226, wherein said promoting agent is EGFR.

243. The Arrangement of claim 226, wherein said promoting agent is MLLT11.

244. The Arrangement of claim 226, wherein said promoting agent is ERBB2.

245. The Arrangement of claim 226, wherein said promoting agent is ETS1.

246. The Arrangement of claim 226, wherein said promoting agent is CSFIR.

247. The Arrangement of claim 226, wherein said promoting agent is FOS.

248. The Arrangement of claim 226, wherein said promoting agent is FES.

249. The Arrangement of claim 226, wherein said promoting agent is GNAS.

250. The Arrangement of claim 226, wherein said promoting agent is HER2.

251. The Arrangement of claim 226, wherein said promoting agent is FGF3.

252. The Arrangement of claim 226, wherein said promoting agent is FGF4.

253. The Arrangement of claim 226, wherein said promoting agent is JUN.

254. The Arrangement of claim 226, wherein said promoting agent is c-kit.

255. The Arrangement of claim 226, wherein said promoting agent is K-SAM.

256. The Arrangement of claim 226, wherein said promoting agent is AKAP13.

257. The Arrangement of claim 226, wherein said promoting agent is LCK.

258. The Arrangement of claim 226, wherein said promoting agent is LM01.

259. The Arrangement of claim 226, wherein said promoting agent is LYL1.

260. The Arrangement of claim 226, wherein said promoting agent is MAS1.

261. The Arrangement of claim 226, wherein said promoting agent is MDM2.

262. The Arrangement of claim 226, wherein said promoting agent is MOS.

263. The Arrangement of claim 226, wherein said promoting agent is MYH11.

264. The Arrangement of claim 226, wherein said promoting agent is MYB.

265. The Arrangement of claim 226, wherein said promoting agent is MYCN.

266. The Arrangement of claim 226, wherein said promoting agent is PAX5.

267. The Arrangement of claim 226, wherein said promoting agent is RAF.

268. The Arrangement of claim 226, wherein said promoting agent is RAS.

269. The Arrangement of claim 226, wherein said promoting agent is REL.

270. The Arrangement of claim 226, wherein said promoting agent is ROS1.

271. The Arrangement of claim 226, wherein said promoting agent is SKI (PDGF-BB).

272. The Arrangement of claim 226, wherein said promoting agent is SET. 273. The Arrangement of claim 226, wherein said promoting agent is SRC.

274. The Arrangement of claim 226, wherein said promoting agent is TAL1.

275. The Arrangement of claim 226, wherein said promoting agent is TAN1.

276. The Arrangement of claim 226, wherein said promoting agent is TIAN.

277. The Arrangement of claim 226, wherein said promoting agent is TSC2.

278. The Arrangement of claim 226, wherein said promoting agent is TRK.

279. The Arrangement of claims 227 to 274, wherein said promoting agent can be conditionally inactivated.

### EXAMPLES

### Example 1: Superior Potency of ToleroChymal Cells as Compared to Mesenchymal Stem Cells at Suppressing Colitis Causing T Cell Proliferation

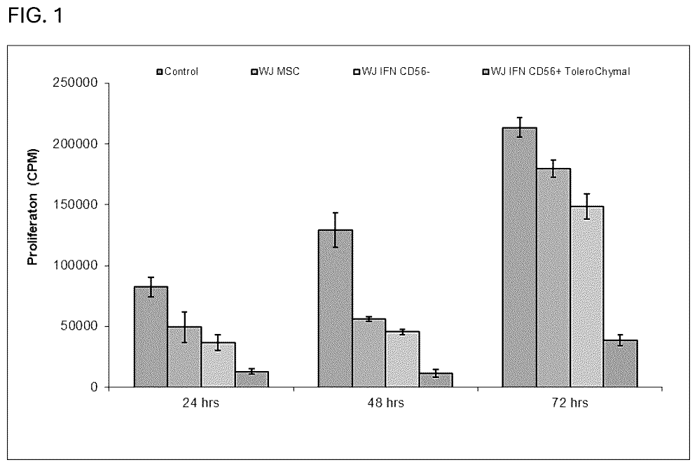

In this non-limiting example, Wharton's Jelly mesenchymal stem cells were isolated by collagenase digestion and plastic adherence. Cells were cultured for 2 passages and exposed to 20 IU of interferon gamma for 24 hours. Selection for CD56 expressing cells was performed by Magnetic Activated Sorting. Assessment of cells to control pathological T cell proliferation (Th17 autoreactive clone) was compared in response to: a) no cells (control); b) Standard WJ Mesenchymal Stem cell; c) CD56 negative fraction after interferon stimulation; and CD56 positive fraction (Tolerochymal Stem Cell) (FIG. 1 and Tables 1-3). Pathological T cells were stimulated with antigen and incubated with stem cells for 24, 48 or 72 hours. The T cell to stem cell ratio was 1 to 1. Proliferation was assessed using the tritiated thymidine incorporation method and expressed as counts per minute (CPM).

TABLE 1
Raw Numbers for 24 Hour Time Point in FIG. 1

| | 24 | | | |
|---|---|---|---|---|
| | hours | WJ | WJ IFN | WJ IFN CD56+ |
| | Control | MSC | CD56- | ToleroChymal |
| 1.0 | 75455.0 | 45355.0 | 57455.0 | 12312.0 |
| 2.0 | 75643.0 | 43456.0 | 32343.0 | 18322.0 |
| 3.0 | 86463.0 | 46543.0 | 22532.0 | 13222.0 |
| 4.0 | 78634.0 | 46342.0 | 22135.0 | 14212.0 |
| 5.0 | 69954.0 | 48542.0 | 29243.0 | 11576.0 |
| 6.0 | 86453.0 | 43654.0 | 23124.0 | 12243.0 |
| 7.0 | 78646.0 | 48543.0 | 43453.0 | 11832.0 |
| 8.0 | 88634.0 | 54354.0 | 45643.0 | 12431.0 |
| 9.0 | 96356.0 | 55436.0 | 44265.0 | 12422.0 |
| 10.0 | 88543.0 | 63563.0 | 48542.0 | 11241.0 |
| Average | 82478.1 | 49578.8 | 36873.5 | 12981.3 |
| STDEV | 8036.8 | 6367.8 | 12584.3 | 2055.6 |

TABLE 2
Raw Numbers for 48 Hour Time Point in FIG. 1

| | 48 hours | WJ | WJ IFN | WJ IFN CD56+ |
|---|---|---|---|---|
| | Control | MSC | CD56- | ToleroChymal |
| 1.0 | 123124.0 | 53455.0 | 43544.0 | 9845.0 |
| 2.0 | 143534.0 | 55545.0 | 44365.0 | 12112.0 |
| 3.0 | 111242.0 | 54355.0 | 44634.0 | 10032.0 |
| 4.0 | 124242.0 | 54775.0 | 47543.0 | 11194.0 |
| 5.0 | 124353.0 | 54763.0 | 43243.0 | 19321.0 |
| 6.0 | 121533.0 | 57689.0 | 44364.0 | 10334.0 |
| 7.0 | 154342.0 | 56456.0 | 48543.0 | 11211.0 |
| 8.0 | 132351.0 | 55476.0 | 47544.0 | 8545.0 |
| 9.0 | 143252.0 | 59646.0 | 47535.0 | 9545.0 |
| 10.0 | 112153.0 | 60123.0 | 44863.0 | 12112.0 |
| Average | 129012.6 | 56228.3 | 45617.8 | 11425.1 |
| STDEV | 14148.0 | 2249.1 | 1951.3 | 2997.1 |

TABLE 3
Raw Numbers for 72 Hour Time Point in FIG. 1

| | 72 hours | | WJ IFN | WJ IFN CD56+ |
|---|---|---|---|---|
| | Control | WJ MSC | CD56- | ToleroChymal |
| 1.0 | 200132.0 | 184234.0 | 145334.0 | 35454.0 |
| 2.0 | 218442.0 | 175434.0 | 154453.0 | 44535.0 |
| 3.0 | 204332.0 | 177423.0 | 145346.0 | 41765.0 |
| 4.0 | 203242.0 | 185343.0 | 134337.0 | 31252.0 |
| 5.0 | 221222.0 | 183242.0 | 154645.0 | 41367.0 |
| 6.0 | 214333.0 | 175435.0 | 144763.0 | 42364.0 |
| 7.0 | 214576.0 | 156434.0 | 158767.0 | 40973.0 |
| 8.0 | 215462.0 | 185343.0 | 154266.0 | 38765.0 |
| 9.0 | 221431.0 | 195788.0 | 147632.0 | 35765.0 |
| 10.0 | 221943.0 | 175354.0 | 147544.0 | 33256.0 |
| Average | 213511.5 | 179403.0 | 148708.7 | 38549.6 |
| STDEV | 8109.8 | 10309.1 | 7052.3 | 4386.7 |

### Example 2: Superior Potency of ToleroChymal Cells as Compared to Mesenchymal Stem Cells at Suppressing Inflammatory Cell Infiltration in Colons of Colitis Mouse Model

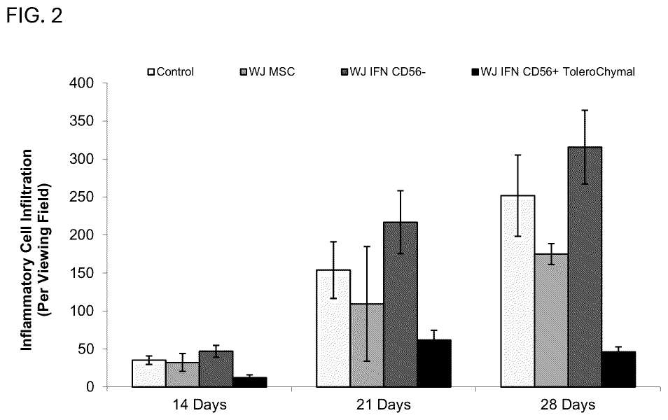

In this non-limiting example, Wharton's Jelly mesenchymal stem cells were isolated by collagenase digestion and plastic adherence. Cells were cultured for 2 passages and exposed to 20 IU of interferon gamma for 24 hours. Selection for CD56 expressing cells was performed by Magnetic Activated Sorting. Assessment of cells to control pathological cell infiltration in colonic tissue was compared in response to: a) no cells (control); b) Standard WJ Mesenchymal Stem cell; c) CD56 negative fraction after interferon stimulation; and CD56 positive fraction (Tolerochymal Stem Cell). Colitis was induced in mice by dextran sulfate sodium (DSS) administration and each of the cell types were administered every second day for a total of 3 injections of 500,000 cells after DSS administration. Mice were sacrificed at days 14, 21 and 28 after DSS administration and inflammatory cells were quantified by H and E staining (FIG. 2 and Tables 4-6).

TABLE 4
Raw Numbers for Day 14 Time Point in FIG. 2.

| | 14 Days | WJ | WJ IFN | WJ IFN CD56+ |
|---|---|---|---|---|
| | Control | MSC | CD56- | ToleroChymal |
| 1.0 | 34.0 | 32.0 | 54.0 | 21.0 |
| 2.0 | 35.0 | 33.0 | 56.0 | 11.0 |
| 3.0 | 43.0 | 35.0 | 54.0 | 15.0 |
| 4.0 | 44.0 | 32.0 | 34.0 | 12.0 |
| 5.0 | 34.0 | 32.0 | 65.0 | 11.0 |
| 6.0 | 35.0 | 12.0 | 53.0 | 8.0 |
| 7.0 | 34.0 | 43.0 | 25.0 | 9.0 |
| 8.0 | 36.0 | 35.0 | 46.0 | 11.0 |
| 9.0 | 35.0 | 32.0 | 42.0 | 14.0 |
| 10.0 | 23.0 | 36.0 | 41.0 | 12.0 |
| Average | 35.3 | 32.2 | 47.0 | 12.4 |
| STDEV | 5.7 | 7.9 | 11.8 | 3.7 |

TABLE 5
Raw Numbers for Day 21 Time Point in FIG. 2.

| | 21 Days | WJ | WJ IFN | WJ IFN CD56+ |
|---|---|---|---|---|
| | Control | MSC | CD56- | ToleroChymal |
| 1.0 | 193.0 | 219.0 | 321.0 | 77.0 |
| 2.0 | 201.0 | 112.0 | 334.0 | 76.0 |
| 3.0 | 143.0 | 125.0 | 231.0 | 77.0 |
| 4.0 | 112.0 | 112.0 | 253.0 | 57.0 |
| 5.0 | 115.0 | 98.0 | 111.0 | 55.0 |
| 6.0 | 212.0 | 89.0 | 143.0 | 74.0 |
| 7.0 | 153.0 | 96.0 | 213.0 | 54.0 |
| 8.0 | 125.0 | 87.0 | 125.0 | 57.0 |
| 9.0 | 121.0 | 77.0 | 223.0 | 45.0 |
| 10.0 | 164.0 | 79.0 | 215.0 | 47.0 |
| Average | 153.9 | 109.4 | 216.9 | 61.9 |
| STDEV | 37.3 | 41.5 | 75.4 | 12.8 |

TABLE 6
Raw Numbers for Day 28 Time Point in FIG. 2.

| | 28 Days | WJ | WJ IFN | WJ IFN CD56+ |
|---|---|---|---|---|
| | Control | MSC | CD56- | ToleroChymal |
| 1.0 | 212.0 | 143.0 | 329.0 | 44.0 |
| 2.0 | 223.0 | 122.0 | 320.0 | 43.0 |
| 3.0 | 214.0 | 143.0 | 323.0 | 46.0 |
| 4.0 | 321.0 | 135.0 | 295.0 | 43.0 |
| 5.0 | 226.0 | 217.0 | 303.0 | 44.0 |
| 6.0 | 235.0 | 204.0 | 312.0 | 46.0 |
| 7.0 | 254.0 | 214.0 | 339.0 | 43.0 |
| 8.0 | 224.0 | 205.0 | 305.0 | 42.0 |
| 9.0 | 375.0 | 114.0 | 305.0 | 45.0 |
| 10.0 | 234.0 | 252.0 | 325.0 | 65.0 |
| Average | 251.8 | 174.9 | 315.6 | 46.1 |
| STDEV | 53.6 | 48.5 | 13.8 | 6.8 |

### Example 3

In this non-limiting example, colitis was induced in mice by dextran sulfate sodium (DSS) administration and the cells prepared as in example 2 (ToleroChymal) were administered every second day for a total of 3 injections of 500,000 cells after DSS administration. Saline and Remicade were also administered.

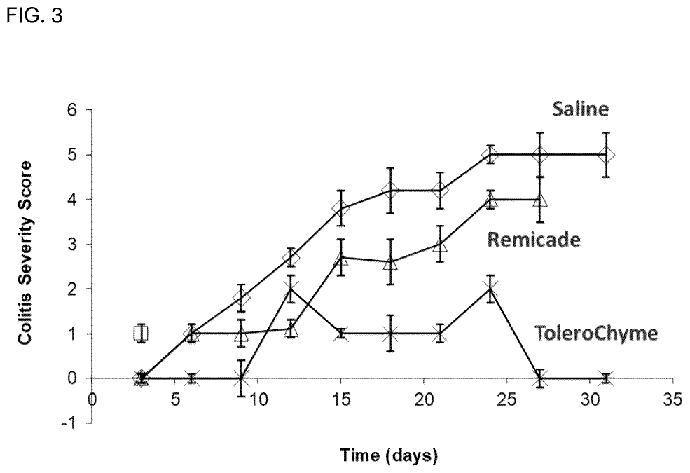

Assessment of colitis severity score was performed according to severity of colitis was assessed daily by scoring the disease activity index (DAI), which includes evaluating stool consistency, body weight loss, and the presence of fecal blood (FIG. 3). Body weight loss was scored as follows: score 0 (no body weight loss), score 1 (body weight loss within 1-5%), score 2 (body weight loss within 5-10%), score 3 (body weight loss within 10-20%), and score 4 (greater than 20% body weight loss). Stool consistency was determined as follows: score 0 (solid pellets), score 1 (soft but adherent in pellet shape), score 2 (loose stool but with some solidity), score 3 (loose stool with signs of liquid consistency), and score 4 (diarrhea). Blood presence in stool was determined as follows: score 0 (no blood), score 2 (red feces), and score 4 (visible bleeding). The score of each parameter was added to yield the final score.

What is claimed is:

1. A method of treating an autoimmune condition by administering mesenchymal stem cells selected for CD56 and subsequently activated with one or more inflammatory stimuli.

2. The method of claim 1, wherein said autoimmune condition is crohn's or colitis.

3. The method of claim 1, wherein said mesenchymal stem cell is derived from a group of tissues comprising of: a) adipose; b) umbilical cord; c) bone marrow; d) pluripotent stem cells; e) dermal; f) omentum; g) menstrual blood; h) mobilized peripheral blood; i) cord blood; j) ovarian; and k) peripheral blood.

4. The method of claim 3, wherein said mobilized peripheral blood is obtained from an individual after administration of one or more agents or procedures that release mesenchymal stem cells into circulation.

5. The method of claim 4, wherein said agent is G-CSF.

6. The method of claim 4, wherein said agent is FLT-3L.

7. The method of claim 4, wherein said agent is ascorbic acid.

8. The method of claim 1, wherein said inflammatory condition is exposure to interleukin-1.

9. The method of claim 1, wherein said inflammatory condition is exposure to interleukin-11.

10. The method of claim 1, wherein said inflammatory condition is exposure to interleukin-23.

11. The method of claim 1, wherein said inflammatory condition is exposure to TNF-alpha.

12. The method of claim 1, wherein said inflammatory condition is exposure to interferon gamma.

13. The method of claim 1, wherein said inflammatory condition is exposure to BlyS

14. The method of claim 1, wherein said inflammatory condition is exposure to interleukin-17.

15. A method of augmenting T regulatory cell activity in an individual suffering from an autoimmune condition wherein said method comprises administering mesenchymal stem cells selected for CD56 and subsequently activated with one or more inflammatory stimuli.

16. The method of claim 15, wherein said T regulatory cells express IL-17 receptor.

17. The method of claim 15, wherein said T regulatory cells express TGF-beta.

18. The method of claim 15, wherein said T regulatory cells express LIF.

19. The method of claim 15, wherein said T regulatory cells express HLA-G.

20. The method of claim 15, wherein said T regulatory cells express FAM13a.
